SEQUENCE LISTING (5 sequences)

SEQ ID NO: 1 - protein, 821 aa, Arabidopsis thaliana
MEMPGRRSNYTLLSQFSDDQVSVSVTGAPPPHYDSLSSENRSNHNSGNTGKAKAERGGFDWDPSGGGGGDHRLNNQPNRVGNNMYASSLGLQRQSSGSSFGESSLSGDYYMPTLSAAANEIESVGFPQDDGFRLGFGGGGGDLRIQMAADSAGGSSSGKSWAQQTEESYQLQLALALRLSSEATCADDPNFLDPVPDESALRTSPSSAETVSHRFWVNGCLSYYDKVPDGFYMMNGLDPYIWTLCIDLHESGRIPSIESLRAVDSGVDSSLEAIIVDRRSDPAFKELHNRVHDISCSCITTKEVVDQLAKLICNRMGGPVIMGEDELVPMWKECIDGLKEIFKVVVPIGSLSVGLCRHRALLFKVLADIIDLPCRIAKGCKYCNRDDAASCLVRFGLDREYLVDLVGKPGHLWEPDSLLNGPSSISISSPLRFPRPKPVEPAVDFRLLAKQYFSDSQSLNLVFDPASDDMGFSMFHRQYDNPGGENDALAENGGGSLPPSANMPPQNMMRASNQIEAAPMNAPPISQPVPNRANRELGLDGDDMDIPWCDLNIKEKIGAGSFGTVHRAEWHGSDVAVKILMEQDFHAERVNEFLREVAIMKRLRHPNIVLFMGAVTQPPNLSIVTEYLSRGSLYRLLHKSGAREQLDERRRLSMAYDVAKGMNYLHNRNPPIVHRDLKSPNLLVDKKYTVKVCDFGLSRLKASTFLSSKSAAGTPEWMAPEVLRDEPSNEKSDVYSFGVILWELATLQQPWGNLNPAQVVAAVGFKCKRLEIPRNLNPQVAAIIEGCWTNEPWKRPSFATIMDLLRPLIKSAVPPPNRSDL

SEQ ID NO: 2 - protein, 821 aa, Artificial sequence
  CTR1ctr1-1-inactive full length CTR1 with D694E mutation
MEMPGRRSNYTLLSQFSDDQVSVSVTGAPPPHYDSLSSENRSNHNSGNTGKAKAERGGFDWDPSGGGGGDHRLNNQPNRVGNNMYASSLGLQRQSSGSSFGESSLSGDYYMPTLSAAANEIESVGFPQDDGFRLGFGGGGGDLRIQMAADSAGGSSSGKSWAQQTEESYQLQLALALRLSSEATCADDPNFLDPVPDESALRTSPSSAETVSHRFWVNGCLSYYDKVPDGFYMMNGLDPYIWTLCIDLHESGRIPSIESLRAVDSGVDSSLEAIIVDRRSDPAFKELHNRVHDISCSCITTKEVVDQLAKLICNRMGGPVIMGEDELVPMWKECIDGLKEIFKVVVPIGSLSVGLCRHRALLFKVLADIIDLPCRIAKGCKYCNRDDAASCLVRFGLDREYLVDLVGKPGHLWEPDSLLNGPSSISISSPLRFPRPKPVEPAVDFRLLAKQYFSDSQSLNLVFDPASDDMGFSMFHRQYDNPGGENDALAENGGGSLPPSANMPPQNMMRASNQIEAAPMNAPPISQPVPNRANRELGLDGDDMDIPWCDLNIKEKIGAGSFGTVHRAEWHGSDVAVKILMEQDFHAERVNEFLREVAIMKRLRHPNIVLFMGAVTQPPNLSIVTEYLSRGSLYRLLHKSGAREQLDERRRLSMAYDVAKGMNYLHNRNPPIVHRDLKSPNLLVDKKYTVKVCEFGLSRLKASTFLSSKSAAGTPEWMAPEVLRDEPSNEKSDVYSFGVILWELATLQQPWGNLNPAQVVAAVGFKCKRLEIPRNLNPQVAAIIEGCWTNEPWKRPSFATIMDLLRPLIKSAVPPPNRSDL

SEQ ID NO: 3 - protein, 288 aa, Artificial sequence
  delta-NT-CTR1
NRELGLDGDDMDIPWCDLNIKEKIGAGSFGTVHRAEWHGSDVAVKILMEQDFHAERVNEFLREVAIMKRLRHPNIVLFMGAVTQPPNLSIVTEYLSRGSLYRLLHKSGAREQLDERRRLSMAYDVAKGMNYLHNRNPPIVHRDLKSPNLLVDKKYTVKVCDFGLSRLKASTFLSSKSAAGTPEWMAPEVLRDEPSNEKSDVYSFGVILWELATLQQPWGNLNPAQVVAAVGFKCKRLEIPRNLNPQVAAIIEGCWTNEPWKRPSFATIMDLLRPLIKSAVPPPNRSDL

SEQ ID NO: 4 - protein, 288 aa, Artificial sequence
  Delta-NT-CTR1ctr1-1 (inactive CTR1 without the N-terminal domain) D694E mutation
NRELGLDGDDMDIPWCDLNIKEKIGAGSFGTVHRAEWHGSDVAVKILMEQDFHAERVNEFLREVAIMKRLRHPNIVLFMGAVTQPPNLSIVTEYLSRGSLYRLLHKSGAREQLDERRRLSMAYDVAKGMNYLHNRNPPIVHRDLKSPNLLVDKKYTVKVCEFGLSRLKASTFLSSKSAAGTPEWMAPEVLRDEPSNEKSDVYSFGVILWELATLQQPWGNLNPAQVVAAVGFKCKRLEIPRNLNPQVAAIIEGCWTNEPWKRPSFATIMDLLRPLIKSAVPPPNRSDL

SEQ ID NO: 5 - protein, 288 aa, Artificial sequence
  delta-NT-CTR1ctr1-1-AAA (inactive CTR1 without the N-terminal domain) T704A/S707A/S710A mutation
NRELGLDGDDMDIPWCDLNIKEKIGAGSFGTVHRAEWHGSDVAVKILMEQDFHAERVNEFLREVAIMKRLRHPNIVLFMGAVTQPPNLSIVTEYLSRGSLYRLLHKSGAREQLDERRRLSMAYDVAKGMNYLHNRNPPIVHRDLKSPNLLVDKKYTVKVCDFGLSRLKASAFLASKAAAGTPEWMAPEVLRDEPSNEKSDVYSFGVILWELATLQQPWGNLNPAQVVAAVGFKCKRLEIPRNLNPQVAAIIEGCWTNEPWKRPSFATIMDLLRPLIKSAVPPPNRSDL

(19) United States
(12) Patent Application Publication
(10) Pub. No.: US 2024/0175042 A1
(43) Pub. Date: May 30, 2024

(54) GENETIC TOOLS USEFUL FOR IMPROVING A PLANT'S STRESS TOLERANCE

(71) Applicant: Purdue Research Foundation, West Lafayette, IN (US)

(72) Inventor: Gyeong Mee YOON, West Lafayette, IN (US)

(73) Assignee: Purdue Research Foundation, West Lafayette, IN (US)

(21) Appl. No.: 17/768,256

(22) PCT Filed: Oct. 16, 2020
(86) PCT No.: PCT/US20/55992

§ 371 Date: Apr. 12, 2022

Related U.S. Application Data
(60) Provisional application No. 62/915,744, filed on Oct. 16, 2019.

(51) Int. Cl. C12N 15/82; C12N 9/12

(52) U.S. Cl. CPC C12N 15/8273; C12N 9/12; C12Y 207/11001

(57) ABSTRACT

This present disclosure relates to methods and composition matters for improving a plant's stress tolerance and a speedy recovery to growth from a stress comprising the process of constitutive expression of a constitutive triple response 1 (CTR1) protein kinase in the nucleus of the cell of said plant. To survive environmental extremes, plants need to rapidly acclimate to stress in order to recover once stress is removed. Here, we uncover a mechanism governing a fast growth recovery of plants following ethylene-induced growth cessation. Ethylene triggers translocation of the CONSTITUTIVE TRIPLE RESPONSE1 (CTR1) protein kinase, a negative regulator of ethylene signaling, from the endoplasmic reticulum (ER) to the nucleus. Nuclear-localized CTR1 inhibits the transcriptional activity of ETHYLENE-INSENSITIVE3 (EIN3) in a kinase independent manner, resulting in rapid reset of the ethylene response, thereby promoting fast growth recovery.

### CROSS REFERENCE TO RELATED APPLICATION

This present patent application relates to and claims the priority under 35 U.S.C. § 119(e) to U.S. Provisional Patent Application Ser. No. 62/915,744 filed on Oct. 16, 2019, the content of which is hereby incorporated by reference in its entirety into the present disclosure.

### GOVERNMENT SUPPORT CLAUSE

This invention was made with government support under MCB1817286 awarded by the National Science Foundation. The government has certain right in the invention.

### STATEMENT OF SEQUENCE LISTING

A computer-readable form (CRF) of the Sequence Listing is submitted with this application. The file, entitled 68822-02_Seq_Listing_ST25_txt, is generated on Sep. 29, 2020. Applicant states that the content of the computer-readable form is the same and the information recorded in computer readable form is identical to the written sequence listing.

### TECHNICAL FIELD

The present invention relates to a method of treatment for chronic pain, opioid dependence, alcohol use disorder or autism. Particularly,

[symbol]

### BACKGROUND

This section introduces aspects that may help facilitate a better understanding of the disclosure. Accordingly, these statements are to be read in this light and are not to be understood as admissions about what is or is not prior art.

The ability of organisms to respond to and to integrate environmental signals to realize an appropriate response is critical for optimal growth and development, particularly for plants, which are non-motile. Plants adapt to a wide variety of abiotic stresses, and upon removal of stress, need rapidly restore basal cellular homeostasis. One key signal for abiotic stress is the plant hormone ethylene. Ethylene is involved in multiple aspects of growth and development, including fruit ripening, leaf and floral senescence, cell elongation, seedling germination, and root hair formation, as well as the response to biotic and abiotic stress [1-3]. Ethylene-mediated stress acclimation includes, but is not limited to, the rapid elongation of rice internodes in response to flooding, drought responses, salt tolerance, heavy metal tolerance, and morphological changes of roots in response to nutrient deficiency [4-7]. However, how ethylene regulates such remarkable plasticity of plant stress adaptation are poorly understood.

Extensive molecular genetics studies have elucidated the basic ethylene signaling pathway [8]. In the absence of ethylene, the endoplasmic reticulum (ER)-localized ethylene receptors activate the constitutive triple response 1 (CTR1) protein kinase, which in turn phosphorylates Ethylene-Insensitive 2 (EIN2), an ER membrane-localized Nramp homolog that positively regulates ethylene responses, to block its cleavage and activation by an unknown protease [8-15]. The CTR1 protein kinase is an important negative regulator of ethylene signaling. CTR1 encodes a serine/threonine (Ser/Thr) protein kinase with an N-terminal regulatory domain and a C-terminal kinase domain. CTR1 acts downstream of the ethylene receptors and upstream of EIN2. When the receptors perceive ethylene, CTR1 kinase activity is shut off, and thereby leading to responses.

In response to ethylene, the receptors, and hence CTR1, are inactive, leading to proteolytic cleavage of EIN2, the C-terminal domain of which (EIN2-CEND) then translocates into the nucleus where it activates Ethylene-Insensitive 3 and its paralogs (EIN3/EIL), master transcription factors in ethylene signaling, to regulate ethylene-responsive gene expression [12, 14, 15]. EIN2-CEND also associates with the EIN3-Binding F-box 1 (EBF1) and EBF2 mRNAs and represses their translation, thus blocking the degradation of EIN3/EIL protein [16, 17]. The function of CTR1 beyond its phosphorylation of EIN2 at the ER has not been characterized.

### BRIEF DESCRIPTION OF DRAWINGS

FIGS. 1A-1H demonstrate ethylene activates CTR1 translocation from ER to the nucleus and EIN2 and EIN3 are not required for this process. (FIG. 1A) Cartoon of CTR1 protein domain structure. (FIG. 1B) WT genomic CTR1 fragment fully rescues ctr1-2 and confers ethylene response. Seedlings were grown for 3 days in the dark with or without ACC or ethylene. MS, Murashige and Skoog medium. Scale bar, 5 mm (FIG. 1C) CTR1 translocates from ER to nucleus in response to ethylene. ER-RK, a mCherry-fused ER marker. (FIG. 1D) GFP-CTR1 fluorescence overlaps with Hoechst nuclear staining under ACC treatment, showing CTR1 nuclear localization. (FIG. 1E) Constitutive nuclear localization of CTR1 in 5-d-old light-grown seedlings. (FIG. 1F) Overexpression of CTR1 leads to constitutive nuclear localization of CTR1 in dark-grown seedlings. (FIG. 1G) EIN2 and EIN3/EIL1 are not required for ACC-induced CTR1 nuclear translocation. (FIG. 1H) Time-lapsed confocal images of a series of hypocotyl cells expressing GFP-CTR1 in 3-d-old etiolated seedlings exposed to 200 μM ACC. Arrows track specific cell nuclei, showing the accumulation of GFP-CTR1 in response to ethylene. All scale bars, 50 μM except B.

FIGS. 2A-2E show that the N-terminus of CTR1 inhibits ACC-induced CTR1 nuclear movement. (FIG. 2A) CTR1 kinase domain does not rescue ctr1-2. (FIG. 2B) Quantitative gene expression analysis for the ethylene-responsive EBF1 in ctr1-2 and CTR1-KD seedlings. (FIG. 2C) Constitutive nuclear localization of GFP-CTR1-KD in hypocotyls of dark-grown seedlings. (FIG. 2D) Fluorescence of GFP-CTR1^{ctr1-8} overlaps with mCherry-ER marker at ER. (FIG. 2E) GFP-CTR1^{ctr1-8} does not translocate to the nucleus in response to ACC or under light conditions.

FIGS. 3A-3E demonstrate that the kinase activity of CTR1 is not necessary for ACC-induced CTR1 nuclear translocation. (FIG. 3A) inactive CTR1 does not rescue ctr1-2. (FIG. 3B) ACC activates nuclear movement of inactive full-length CTR1^{dead} and inactive CTR1-KD^{dead} in etiolated seedlings. (FIG. 3C) The constitutive nuclear localization of CTR1 kinase domain with the ctr1-1 mutation in etiolated seedlings. (FIG. 3D) Phos-tag analysis of CTR1-KD and CTR1-KD^{AAA}, showing no in vivo kinase activity of CTR1-KD^{AAA}. EIN2-CEND with co-expression with CTR1-KD showed a shifted band in phos-tag gel, indicating a phosphorylation by the CTR1-KD, but not in SDS-PAGE. (FIG. 3E) The mutations of autophosphorylation sites in CTR1 activation loop do not inhibit the constitutive nuclear localization of CTR1-KD^{AAA}.

FIGS. 4A-4C show the nuclear-localized CTR-mediated suppression on growth inhibition during the phase I plateau and faster growth recovery. Elongation kinetics of 2-d-old etiolated Arabidopsis hypocotyls in response to ethylene for WT and seedlings expressing CTR1-KD, CTR1-KD^{dead}, CTR1, CTR1^{dead} or CTR1^{ctr1-8}, and ctr1-8 mutant seedlings. Hypocotyl growth rate was recorded for 1 h in air, followed by 2 h exposure to 10 ppm ethylene, and then a 5 h recovery in air. Graphs in the left panel show the full-time course and Graphs in the right show an enlargement of response immediately after ethylene exposure. The responses of wild-type seedlings (▪) are shown in each graph. (FIG. 4A) 35Sp:GFP-CTR1-KD and 35Sp:GFP-CTR1-KD^{dead}. (FIG. 4B) 35Sp:GFP-CTR1 and 35Sp:GFP-CTR1^{dead}. (FIG. 4C) ctr1-8 and 35Sp:GFP-CTR1^{ctr1-8}.

FIG. 5 demonstrates ethylene response of transgenic lines overexpressing various CTR1 transgenes. Representative images of etiolated seedlings of WT and transgenic lines overexpressing CTR1 or its variants were grown in MS medium with or without ACC for 3-d. Scale bar, 5 mm.

FIGS. 6A-6B show the comparison of growth kinetics of etiolated Arabidopsis hypocotyls expressing wild type CTR1 or CTR1 mutant protein in response to ethylene. Growth rates were recorded for 1 h in air followed by a 2 h exposure to 10 mL/L ethylene. This was followed by 5 h in air. The responses of wild-type Col-0 hypocotyls are shown in closed squares for comparison with 35S:GFP-CTR1 or 35S:GFP-CTR1-KD (FIG. 6A) and 35S:GFP-CTR1^{dead} (closed green diamond) or 35S:GFP-CTR1-KD^{dead} (open red triangle) (FIG. 6B). All data represent averages of at least 50 seedlings±SE.

FIGS. 7A-7C show that the nuclear-localized CTR1-mediated suppression on growth inhibition during the phase I plateau. Elongation kinetics of 2-d-old etiolated Arabidopsis hypocotyls in response to ethylene for WT and seedlings expressing CTR1-KD, CTR1-KD^{dead} (FIG. 7A), CTR1, CTR1^{dead} (FIG. 7B), or CTR1^{ctr1-8}, and ctr1-8 mutant (FIG. 7C) seedlings. Hypocotyl growth rate was recorded for 1 h in air, followed by 2 h exposure to 10 ppm ethylene, and then a 5 h recovery in air. Graphs show an enlargement of response immediately after ethylene exposure. The dotted rectangles indicate the phase I plateau. The responses of wild-type seedlings (▪) are shown in each graph. All data represent averages of at least 50 seedlings±SE.

FIGS. 8A-8E show that nuclear-localized CTR1 downregulates EIN3 via a non-catalytic function. (FIG. 8A) Schematic diagrams of the effector, reporter, and internal control plasmids used in the transient transactivation assay in Arabidopsis leaf protoplasts. (FIGS. 8B-8C) Transactivation of the Luc reporter gene by EIN3 and full-length WT CTR1 (FIG. 8B) or CTR1-KD^{ctr1-1} (FIG. 8C) with (green) or without ACC (dark blue) in Arabidopsis protoplasts. (FIG. 8D) BiFC assay for full-length WT CTR1 and nuclear ethylene signaling proteins in N. benthamiana in the presence of ethylene. (FIG. 8E) CTR1-KD^{ctr1-1} promotes the degradation of EIN3. Indicated plasmids were co-transfected to Arabidopsis protoplasts and incubated for 16 h and the protoplasts were subsequently incubated with ACC for additional 2 h followed by total protein extraction and western blotting using anti-GFP antibody.

FIGS. 9A-9B show in vitro kinase assay for EBF2 by CTR1. (FIG. 9A) Coomassie-stained SDS/PAGE gel of purified EIN2-CEND, EIN3, and EBF2. Molecular weight markers are shown on left. (FIG. 9B) In vitro kinase assay of purified CTR1-KD or CTR1-KD^{dead} (residues 531-821) with the EIN2-CEND, EIN3, or EBF2. EIN2-CEND and EIN3 were used as positive and negative control, respectively. The indicated proteins were incubated together in kinase reaction buffer, separated by SDS/PAGE, and the incorporated radiolabel detected.

FIG. 10 depicts a model for ethylene-induced CTR1 nuclear translocation and suppression of ethylene response. In the absence of ethylene, CTR1 localizes to the ER and phosphorylates EIN2, leading to the proteolytic degradation of EIN2 via EIN2-Targeting Proteins (ETP1/2). Upon the perception of ethylene by the ethylene receptors, inactivated CTR1 no longer phosphorylates EIN2, resulting in proteolytic cleavage of the C-terminal domain of EIN2 (EIN2-CEND). EIN2-CEND is subsequently translocated to the nucleus for EIN3 activation or to processing body (P-body) to suppress EBFs mRNA translation. Likewise, in the presence of ethylene, CTR1 is released from the receptor, which stimulates the nuclear translocation of CTR1 via an unknown mechanism. The nuclear-localized CTR1 activates EBFs via the direct interaction resulting in promotion of EIN3 degradation. The tight regulation on controlling equilibrium between EIN2-mediated EIN3 activation and CTR1-mediated EIN3 inhibition in the presence of ethylene is required for fine-tuning of ethylene response during stress acclimation and for rapid suppression of ethylene response when the stress is removed. Yellow square with a question mark indicates an unknown cargo protein that delivers CTR1 to the nucleus. Arrows indicate movement direction or positive influence; blunted ends indicate inhibition.

FIGS. 11A-11E demonstrate faster growth recovery kinetics confers stress tolerance. WT, CTR1-KD^{dead}, and CTR1-KD were used for analysis. FIG. 11A shows the long-term effects of drought on WT and two CTR1 lines. Two weeks old WT and CTR1-KD^{ctr1-1} and CTR1-KD seedlings were subjected for water stress by withdrawing water for 28-d, followed by 8-d re-watering. FIG. 11B shows the seedling growth on MS media containing 175 mM NaCl for 2-weeks. FIG. 11C shows the survival rate of seedlings on MS media with 175 mM NaCl in FIG. 11B. FIGS. 11D-11E demonstrate the effects of salt on seedlings on WT and CTR1-KD^{dead} and CTR1-KD. Two-weeks-old seedlings were irrigated with 200 or 300 mM NaCl solution for 28-d (FIG. 11D), then recovered for 8-d (FIG. 11E).

### BRIEF DESCRIPTION ON THE RELATED SEQUENCE LISTING

SEQ ID NO: 1, Full length CTR1 (at5g03730)
MEMPGRRSNYTLLSQFSDDQVSVSVTGAPPPHYDSLSSENRSNHNSGNTGKAKAERGGFD
WDPSGGGGGDHRLNNQPNRVGNNMYASSLGLQRQSSGSSFGESSLSGDYYMPTLSAAANE
IESVGFPQDDGFRLGFGGGGGDLRIQMAADSAGGSSSGKSWAQQTEESYQLQLALALRLS
SEATCADDPNFLDPVPDESALRTSPSSAETVSHRFWVNGCLSYYDKVPDGFYMMNGLDPY
IWTLCIDLHESGRIPSIESLRAVDSGVDSSLEAIIVDRRSDPAFKELHNRVHDISCSCIT
TKEVVDQLAKLICNRMGGPVIMGEDELVPMWKECIDGLKEIFKVVVPIGSLSVGLCRHRA
LLFKVLADIIDLPCRIAKGCKYCNRDDAASCLVRFGLDREYLVDLVGKPGHLWEPDSLLN
GPSSISISSPLRFPRPKPVEPAVDFRLLAKQYFSDSQSLNLVFDPASDDMGFSMFHRQYD
NPGGENDALAENGGGSLPPSANMPPQNMMRASNQIEAAPMNAPPISQPVPNRANRELGLD
GDDMDIPWCDLNIKEKIGAGSFGTVHRAEWHGSDVAVKILMEQDFHAERVNEFLREVAIM
KRLRHPNIVLFMGAVTQPPNLSIVTEYLSRGSLYRLLHKSGAREQLDERRRLSMAYDVAK
GMNYLHNRNPPIVHRDLKSPNLLVDKKYTVKVCDFGLSRLKASTFLSSKSAAGTPEWMAP
EVLRDEPSNEKSDVYSFGVILWELATLQQPWGNLNPAQVVAAVGFKCKRLEIPRNLNPQV
AAIIEGCWTNEPWKRPSFATIMDLLRPLIKSAVPPPNRSDL
SEQ ID NO: 2, CTR1^{ctr1-1}-inactive full length CTR1 with D694E mutation
MEMPGRRSNYTLLSQFSDDQVSVSVTGAPPPHYDSLSSENRSNHNSGNTGKAKAERGGFD
WDPSGGGGGDHRLNNQPNRVGNNMYASSLGLQRQSSGSSFGESSLSGDYYMPTLSAAANE
IESVGFPQDDGFRLGFGGGGGDLRIQMAADSAGGSSSGKSWAQQTEESYQLQLALALRLS
SEATCADDPNFLDPVPDESALRTSPSSAETVSHRFWVNGCLSYYDKVPDGFYMMNGLDPY
IWTLCIDLHESGRIPSIESLRAVDSGVDSSLEAIIVDRRSDPAFKELHNRVHDISCSCIT
TKEVVDQLAKLICNRMGGPVIMGEDELVPMWKECIDGLKEIFKVVVPIGSLSVGLCRHRA
LLFKVLADIIDLPCRIAKGCKYCNRDDAASCLVRFGLDREYLVDLVGKPGHLWEPDSLLN
GPSSISISSPLRFPRPKPVEPAVDFRLLAKQYFSDSQSLNLVFDPASDDMGFSMFHRQYD
NPGGENDALAENGGGSLPPSANMPPQNMMRASNQIEAAPMNAPPISQPVPNRANRELGLD
GDDMDIPWCDLNIKEKIGAGSFGTVHRAEWHGSDVAVKILMEQDFHAERVNEFLREVAIM
KRLRHPNIVLFMGAVTQPPNLSIVTEYLSRGSLYRLLHKSGAREQLDERRRLSMAYDVAK
GMNYLHNRNPPIVHRDLKSPNLLVDKKYTVKVCEFGLSRLKASTFLSSKSAAGTPEWMAP
EVLRDEPSNEKSDVYSFGVILWELATLQQPWGNLNPAQVVAAVGFKCKRLEIPRNLNPQV
AAIIEGCWTNEPWKRPSFATIMDLLRPLIKSAVPPPNRSDL
SEQ ID NO: 3, ΔNT-CTR1
NRELGLDGDDMDIPWCDLNIKEKIGAGSFGTVHRAEWHGSDVAVKILMEQDFHAERVNEFLREVAIM
KRLRHPNIVLFMGAVTQPPNLSIVTEYLSRGSLYRLLHKSGAREQLDERRRLSMAYDVAK
GMNYLHNRNPPIVHRDLKSPNLLVDKKYTVKVCDFGLSRLKASTFLSSKSAAGTPEWMAP
EVLRDEPSNEKSDVYSFGVILWELATLQQPWGNLNPAQVVAAVGFKCKRLEIPRNLNPQV
AAIIEGCWTNEPWKRPSFATIMDLLRPLIKSAVPPPNRSDL
SEQ ID NO: 4, ΔNT-CTR1^{ctr1-1} (inactive CTR1 without the N-terminal domain):
D694E mutation
NRELGLDGDDMDIPWCDLNIKEKIGAGSFGTVHRAEWHGSDVAVKILMEQDFHAERVNEFLREVAIM
KRLRHPNIVLFMGAVTQPPNLSIVTEYLSRGSLYRLLHKSGAREQLDERRRLSMAYDVAK
GMNYLHNRNPPIVHRDLKSPNLLVDKKYTVKVCEFGLSRLKASTFLSSKSAAGTPEWMAP
EVLRDEPSNEKSDVYSFGVILWELATLQQPWGNLNPAQVVAAVGFKCKRLEIPRNLNPQV
AAIIEGCWTNEPWKRPSFATIMDLLRPLIKSAVPPPNRSDL
SEQ ID NO: 5, ΔNT-CTR1^{ctr1-1-AAA} (inactive CTR1 without the N-terminal domain):
T704A/S707A/S710A mutation
NRELGLDGDDMDIPWCDLNIKEKIGAGSFGTVHRAEWHGSDVAVKILMEQDFHAERVNEFLREVAIM
KRLRHPNIVLFMGAVTQPPNLSIVTEYLSRGSLYRLLHKSGAREQLDERRRLSMAYDVAK
GMNYLHNRNPPIVHRDLKSPNLLVDKKYTVKVCDFGLSRLKASTFLSSKSAAGTPEWMAP
EVLRDEPSNEKSDVYSFGVILWELATLQQPWGNLNPAQVVAAVGFKCKRLEIPRNLNPQV
AAIIEGCWTNEPWKRPSFATIMDLLRPLIKSAVPPPNRSDL

Plants overexpressing a CTR1 mutant, a fragment, or an analog protein have an increased stress resistance to drought, salinity, and other environmental stresses, as well as a speedier recovery from those stresses to growth. A non-exclusive, illustrative list of example proteins are:
1) SEQ ID NO: 2, CTR1^{ctr1-1}-inactive full length CTR1 with D694E mutation;
2) SEQ ID NO: 3, ΔNT-CTR1;
3) SEQ ID NO: 4, ΔNT-CTR1^{ctr1-1} (inactive CTR1 without the N-terminal domain): D694E mutation; and
4) SEQ ID NO: 5, ΔNT-CTR1^{ctr1-1-AAA} (inactive CTR1 without the N-terminal domain): T704A/S707A/S710A mutation.

### DETAILED DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure, the term “substantially” can allow for a degree of variability in a value or range, for example, within 90%, within 95%, 99%, 99.5%, 99.9%, 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

To survive environmental extremes, plants need to rapidly acclimate to stress in order to recover once stress is removed. Here, we uncover a mechanism governing a fast growth recovery of plants following ethylene-induced growth cessation. Ethylene triggers translocation of the CONSTITUTIVE TRIPLE RESPONSE1 (CTR1) protein kinase, a negative regulator of ethylene signaling, from the endoplasmic reticulum (ER) to the nucleus. Nuclear-localized CTR1 inhibits the transcriptional activity of ETHYLENE-INSENSITIVE3 (EIN3) in a kinase independent manner, resulting in rapid reset of the ethylene response, thereby promoting fast growth recovery. These findings reveal a novel mechanism linking the spatiotemporal dynamics of cellular signaling components to organismal stress response.

It is our understanding that CTR1 localizes in the Endoplasmic Reticulum (ER) and moves to the nucleus when the ethylene signaling pathway is activated by stresses such as drought and salinity stress (CTR1 is not expressed in the nucleus per se). The movement of CTR1 from ER to the nucleus results in attenuating nuclear ethylene response, which promotes the rapid recovery of plants when stress is being withdrawn. To the best of our knowledge, most plants that have functional ethylene signaling pathway have this CTR1 pathway to deal with environmental stresses and a speedy recovery to growth.

This present disclosure relates to methods and composition matters for improving a plant's stress tolerance and a speedier recovery to growth from a stress comprising the process of constitutive expression of a constitutive triple response 1 (CTR1) protein kinase in the nucleus of the cell of said plant.

In some illustrative embodiments, this disclosure relates to a method of boosting the stress tolerance to drought, salt, and other environmental stresses, as well as a speedier recovery from those stresses, by overexpression of an analog, a fragment, or a mutant of the natural CTR1 in the nucleus.

In some illustrative embodiments, this disclosure relates to a method of boosting the stress tolerance to drought, salt, and other environmental stresses, as well as a speedier recovery from those stresses, by overexpression of an analog, a fragment, or a mutant of the natural CTR1 in the nucleus, wherein the deletion of the N-terminal domain of CTR1 results in a constitutive nuclear localization of CTR1, therefore plants always express the mutant form of CTR1 in the nucleus, giving more stress tolerance. Full-length CTR1 (no N-terminal deletion) also moves to the nucleus when it overexpressed in plants, but not at the same levels as the delta N-CTR1.

In some illustrative embodiments, this disclosure relates to a method of boosting the stress tolerance to drought, salt, and other environmental stresses, as well as a speedier recovery from those stresses, by overexpression of an analog, a fragment, or a mutant of the natural CTR1 in the nucleus, wherein the protein kinase activity of CTR1 does not influence of CTR1 nuclear movement, but overexpression of CTR1, whether full-length or with the N-terminal domain removed (delta N), confers a much increased stress tolerance drought, salt, and other environmental stresses.

In some illustrative embodiments, this disclosure relates to a method of boosting the stress tolerance to drought, salt, and other environmental stresses, as well as a speedier recovery from those stresses, by overexpression of an analog, a fragment, or a mutant of the natural CTR1 in the nucleus, wherein the protein kinase activity of CTR1 does not influence of CTR1 nuclear movement, but overexpression of CTR1, whether full-length or with the N-terminal domain removed (delta N), confers a much increased stress tolerance to drought, salt, and other environmental stresses, while the CTR1, a mutant, or a fragment thereof has no kinase activity.

In some illustrative embodiments, this disclosure relates to a method of boosting the stress tolerance to drought, salt, and other environmental stresses, as well as a speedier recovery from those stresses, by overexpression of an analog, a fragment, or a mutant of the natural CTR1 in the nucleus, wherein the protein kinase activity of CTR1 does not influence of CTR1 nuclear movement, but overexpression of CTR1, whether full-length or with the N-terminal domain removed (delta N), confers a much increased stress tolerance to physical and environmental stresses, such as drought, salt, flooding (submergence), freezing, chilling, extreme temperature (cold, frost, heat), radiation, and biotic stresses caused by a living organism, such as virus, bacterium, fungus, nematode, insect, or arachnid.

In some illustrative embodiments, this disclosure relates to a method for improving a plant's stress tolerance and a speedy recovery to growth from said stress by promoting constitutive expression of an analog, a fragment, or a mutant of the constitutive triple response 1 (CTR1) protein kinase (SEQ ID NO: 1) in the cell nucleus of said plant.

In some illustrative embodiments, this disclosure relates to a method for improving a plant's stress tolerance and a speedy recovery to growth from said stress by promoting constitutive expression of an analog, a fragment, or a mutant of the constitutive triple response 1 (CTR1) protein kinase (SEQ ID NO: 1) in the cell nucleus of said plant as disclosed herein, wherein said analog, fragment, or mutant of CTR1 protein kinase (SEQ ID NO: 1) in the cell nucleus of said plant has no protein kinase activity.

In some illustrative embodiments, this disclosure relates to a method for improving a plant's stress tolerance and a speedy recovery to growth from said stress by promoting constitutive expression of an analog, a fragment, or a mutant of the constitutive triple response 1 (CTR1) protein kinase (SEQ ID NO: 1) in the cell nucleus of said plant as disclosed herein, wherein said analog, fragment, or mutant of CTR1 protein kinase (SEQ ID NO: 1) in the cell nucleus of said plant is a fragment of CTR1 with the N-terminal domain removed (delta N).

In some illustrative embodiments, this disclosure relates to a method for improving a plant's stress tolerance and a speedy recovery to growth from said stress by promoting constitutive expression of an analog, a fragment, or a mutant of the constitutive triple response 1 (CTR1) protein kinase (SEQ ID NO: 1) in the cell nucleus of said plant as disclosed herein, wherein said CTR1 protein kinase is an analog of the CTR1 protein kinase (SEQ ID NO: 1).

In some illustrative embodiments, this disclosure relates to a method for improving a plant's stress tolerance and a speedy recovery to growth from said stress by promoting constitutive expression of an analog, a fragment, or a mutant of the constitutive triple response 1 (CTR1) protein kinase (SEQ ID NO: 1) in the cell nucleus of said plant as disclosed herein, wherein said CTR1 protein kinase is a mutant of the CTR1 protein kinase (SEQ ID NO: 1).

In some illustrative embodiments, this disclosure relates to a method for improving a plant's stress tolerance and a speedy recovery to growth from said stress by promoting constitutive expression of an analog, a fragment, or a mutant of the constitutive triple response 1 (CTR1) protein kinase (SEQ ID NO: 1) in the cell nucleus of said plant as disclosed herein, wherein said analog, fragment, or mutant of CTR1 protein kinase (SEQ ID NO: 1) has no protein kinase activity.

In some illustrative embodiments, this disclosure relates to a method for improving a plant's stress tolerance and a speedy recovery to growth from said stress by promoting constitutive expression of an analog, a fragment, or a mutant of the constitutive triple response 1 (CTR1) protein kinase (SEQ ID NO: 1) in the cell nucleus of said plant as disclosed herein, wherein said CTR1 protein kinase is a mutant (D694E) having SEQ ID NO: 2, or a functional analog and/or fragment thereof.

In some illustrative embodiments, this disclosure relates to a method for improving a plant's stress tolerance and a speedy recovery to growth from said stress by promoting constitutive expression of an analog, a fragment, or a mutant of the constitutive triple response 1 (CTR1) protein kinase (SEQ ID NO: 1) in the cell nucleus of said plant as disclosed herein, wherein said CTR1 protein kinase comprises SEQ ID NO: 3, or a functional analog and/or fragment thereof.

In some illustrative embodiments, this disclosure relates to a method for improving a plant's stress tolerance and a speedy recovery to growth from said stress by promoting constitutive expression of an analog, a fragment, or a mutant of the constitutive triple response 1 (CTR1) protein kinase (SEQ ID NO: 1) in the cell nucleus of said plant as disclosed herein, wherein said CTR1 protein kinase mutant comprises SEQ ID NO: 4, or a functional analog and/or fragment thereof.

In some illustrative embodiments, this disclosure relates to a method for improving a plant's stress tolerance and a speedy recovery to growth from said stress by promoting constitutive expression of an analog, a fragment, or a mutant of the constitutive triple response 1 (CTR1) protein kinase (SEQ ID NO: 1) in the cell nucleus of said plant as disclosed herein, wherein said CTR1 protein kinase mutant with T704A, S707A, and S710A mutations, comprises SEQ ID NO: 5, or a functional analog and/or fragment thereof.

In some illustrative embodiments, this disclosure relates to a method for improving a plant's stress tolerance and a speedy recovery to growth from said stress by promoting constitutive expression of an analog, a fragment, or a mutant of the constitutive triple response 1 (CTR1) protein kinase (SEQ ID NO: 1) in the cell nucleus of said plant as disclosed herein, wherein said analog, fragment, or mutant of the CTR1 protein kinase (SEQ ID NO: 1) has its N-terminal domain removed.

In some illustrative embodiments, this disclosure relates to a method for improving a plant's stress tolerance and a speedy recovery to growth from said stress by promoting constitutive expression of an analog, a fragment, or a mutant of the constitutive triple response 1 (CTR1) protein kinase (SEQ ID NO: 1) in the cell nucleus of said plant as disclosed herein, wherein said stress comprises drought, salinity, and other environmental stresses comprising flooding (submergence), freezing, chilling, extreme temperature (cold, frost, heat), radiation, biotic stresses caused by a living organism selected from the group consisting of viruses, bacteria, fungi, nematodes, insects, and arachnids.

In some illustrative embodiments, this disclosure relates to a method for improving a plant's stress tolerance and a speedy recovery to growth from said stress by promoting constitutive expression of an analog, a fragment, or a mutant of the constitutive triple response 1 (CTR1) protein kinase (SEQ ID NO: 1) in the cell nucleus of said plant as disclosed herein, wherein said plant expresses CTR1-like protein kinase and wherein overexpression of the corresponding CTR1-like genes in the nucleus increases said plant's tolerance to physical and environmental stresses.

In some illustrative embodiments, this disclosure relates to a method for improving a plant's stress tolerance and a speedy recovery to growth from said stress by promoting constitutive expression of an analog, a fragment, or a mutant of the constitutive triple response 1 (CTR1) protein kinase (SEQ ID NO: 1) in the cell nucleus of said plant as disclosed herein, wherein said plant comprises soybean, corn, rice, sorghum, potato, wheat, barley, and peanut.

In some other illustrative embodiments, this disclosure relates to an analog, a fragment, or a mutant of CTR1 protein kinase (SEQ ID NO: 1) useful for improving a plant's stress tolerance and a speedy recovery to growth from a stress.

In some other illustrative embodiments, this disclosure relates to an analog, a fragment, or a mutant of CTR1 protein kinase (SEQ ID NO: 1) useful for improving a plant's stress tolerance and a speedy recovery to growth from said stress as disclosed herein, wherein said CTR1 protein kinase mutant comprises a SEQ ID NO: 2, or a functional analog and/or fragment thereof.

In some other illustrative embodiments, this disclosure relates to an analog, a fragment, or a mutant of CTR1 protein kinase (SEQ ID NO: 1) useful for improving a plant's stress tolerance and a speedy recovery to growth from said stress as disclosed herein, wherein said CTR1 protein kinase mutant comprises a SEQ ID NO: 3, or a functional analog and/or fragment thereof.

In some other illustrative embodiments, this disclosure relates to an analog, a fragment, or a mutant of CTR1 protein kinase (SEQ ID NO: 1) useful for improving a plant's stress tolerance and a speedy recovery to growth from said stress as disclosed herein, wherein said CTR1 protein kinase mutant comprises a SEQ ID NO: 4, or a functional analog and/or fragment thereof.

In some other illustrative embodiments, this disclosure relates to an analog, a fragment, or a mutant of CTR1 protein kinase (SEQ ID NO: 1) useful for improving a plant's stress tolerance and a speedy recovery to growth from said stress as disclosed herein, wherein said CTR1 protein kinase mutant comprises a SEQ ID NO: 5, or a functional analog and/or fragment thereof.

In some other illustrative embodiments, this disclosure relates to an analog, a fragment, or a mutant of CTR1 protein kinase (SEQ ID NO: 1) useful for improving a plant's stress tolerance and a speedy recovery to growth from said stress as disclosed herein, wherein said stress comprises both physical and environmental stresses.

In some other illustrative embodiments, this disclosure relates to an analog, a fragment, or a mutant of CTR1 protein kinase (SEQ ID NO: 1) useful for improving a plant's stress tolerance and a speedy recovery to growth from said stress as disclosed herein, wherein said stress comprises drought, salinity, and other environmental stresses comprising flooding (submergence), freezing, chilling, extreme temperature (cold, frost, heat), radiation, biotic stresses caused by a living organism selected from the group consisting of viruses, bacteria, fungi, nematodes, insects, and arachnids.

In some other illustrative embodiments, this disclosure relates to an analog, a fragment, or a mutant of CTR1 protein kinase (SEQ ID NO: 1) useful for improving a plant's stress tolerance and a speedy recovery to growth from said stress as disclosed herein, wherein said analog, fragment, or mutant of the CTR1 protein kinase (SEQ ID NO: 1) has its N-terminal domain removed.

In some other illustrative embodiments, this disclosure relates to an analog, a fragment, or a mutant of CTR1 protein kinase (SEQ ID NO: 1) useful for improving a plant's stress tolerance and a speedy recovery to growth from said stress as disclosed herein, wherein said plant comprises soybean, corn, rice, sorghum, potato, wheat, barley, peanut, and others.

In some other illustrative embodiments, this disclosure relates to an analog, a fragment, or a mutant of CTR1 protein kinase (SEQ ID NO: 1) useful for improving a plant's stress tolerance and a speedy recovery to growth from said stress as disclosed herein, wherein said plant comprises soybean, corn, rice, sorghum, potato, wheat, barley, peanut, and others wherein overexpression of an analog, a fragment, or a mutant of CTR1 protein kinase (SEQ ID NO: 1) is practically applicable.

Dark-grown seedlings exposed to ethylene show dramatic reduction in growth rate. However, following removal of ethylene, the seedlings return to a basal rate of growth within 90 min [18, 19], even though the proteolytic cleavage of EIN2 is irreversible. How the nuclear-localized EIN2-CEND is turned off in the nucleus is an open question. Here, we report that surprisingly, CTR1 translocate from ER to nucleus rapidly in response to ethylene. The nuclear-localized CTR1 rapidly inhibits ethylene response through a mechanism that does not require its kinase activity, and promotes the recovery of seedling growth back to basal levels. Plants overexpressing the kinase domain of CTR1, which constitutively localized to the nucleus, exhibited faster growth recovery kinetics following withdrawal of ethylene and abiotic stress tolerance. These results suggest a new paradigm for the dynamic regulation of the ethylene signaling involving the subcellular localization of CTR1 that influences the ability to tolerate abiotic stress.

### Ethylene-Induced ER-to-Nuclear Translocation of CTR1

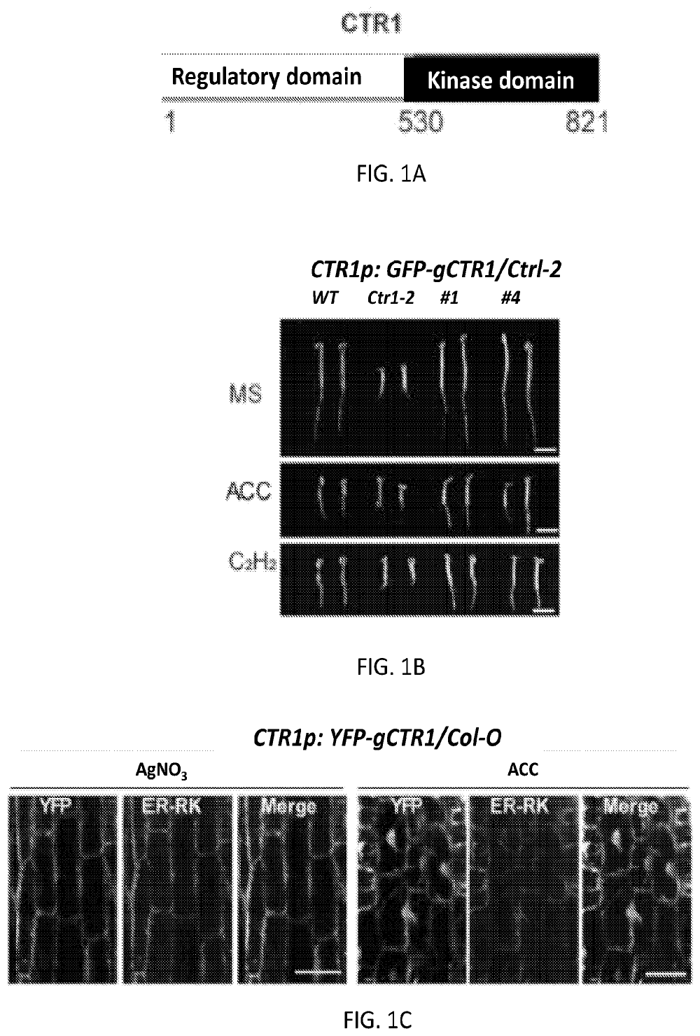

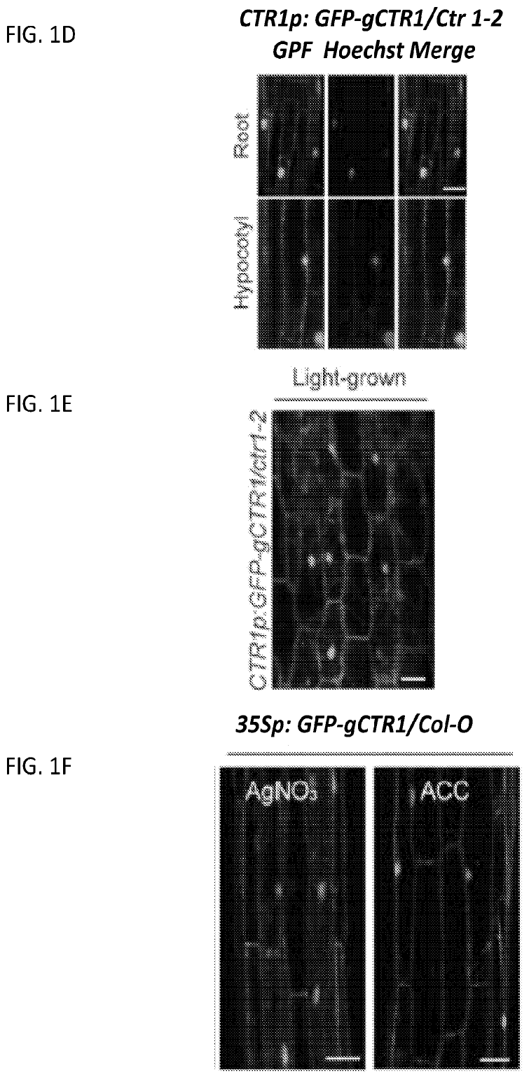

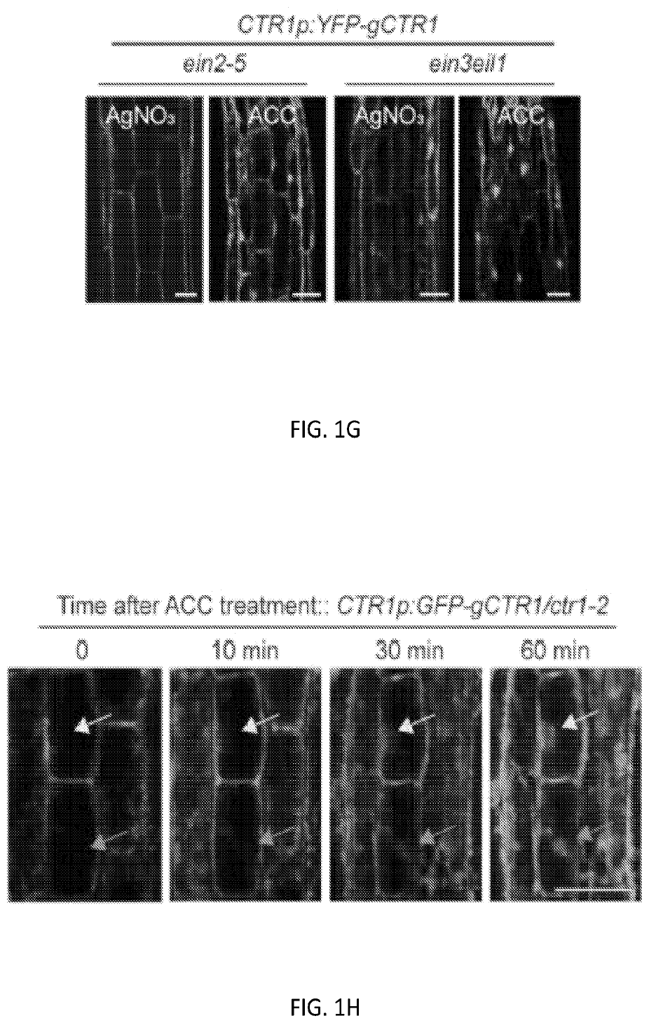

CTR1 consists of an N-terminal regulatory domain and a C-terminal kinase domain that is homologous to the catalytic domain of Raf kinase family [13] (FIG. 1A). To determine the role of CTR1 beyond its role in regulating EIN2, we examined the subcellular localization of CTR1 following exposure to exogenous ethylene. To this end, we introduced ˜7.6 Kb genomic CTR1 transgene including CTR1 promoter region (0.7 kb upstream of the 5′UTR) driving expression of the CTR1 coding region fused to a GFP reporter (CTR1p:GFP-gCTR1) into the ctr1-2 loss-of-function mutant. The CTR1p:GFP-gCTR1 transgene fully complemented the ctr1-2 phenotypes in both light and dark-grown seedlings (FIG. 1B), indicating that this fusion protein is functional. In the absence of ethylene signaling, the CTR1-GFP fusion protein localized to the ER (FIG. 1C), consistent with previous reports [10, 11]. However, surprisingly, in response to exogenous ethylene, CTR1-GFP accumulated in the nucleus (FIGS. 1C and 1D). In contrast to etiolated seedlings, GFP-CTR1 constitutively localized to the nucleus in light-grown seedlings (FIG. 1E), likely due to higher basal ethylene levels in light-grown seedlings. Furthermore, when GFP-CTR1 was expressed under the control of the strong CaMV 35S promoter, it constitutively localized in the nucleus regardless of ACC treatment (FIG. 1F). This could result from limited cellular components required to tether CTR1 to the ER. As both ACC and ethylene caused equivalent CTR1 nuclear translocation, we employed ACC for further studies. Disruption of either EIN2 or EIN3/EIL1 did not prevent the nuclear accumulation of CTR1 (FIG. 1G), suggesting that EIN2 and EIN3 are not required for CTR1 nuclear translocation.

EIN2-CEND migrates into the nucleus within 10 min following ethylene treatment [14]. To determine the nuclear movement kinetics of CTR1 upon ethylene perception, we monitored the dynamics of CTR1 movement in response to ACC treatment. CTR1 first began to accumulate in the nucleus 30 min after ACC treatment, with a further increase in nuclear protein levels after 30 min (FIG. 1H). Overall levels of GFP fluorescence intensity were enhanced with the longer ACC exposure, consistent with increased stabilization of CTR1 protein in response to ACC, similar to previous reports. Together, these results demonstrated that ethylene stimulates the translocation of CTR1 from the ER to the nucleus in an EIN2 and EIN3/EIL independent manner.

### The CTR1 N-Terminus Inhibits CTR1 Nuclear Trafficking

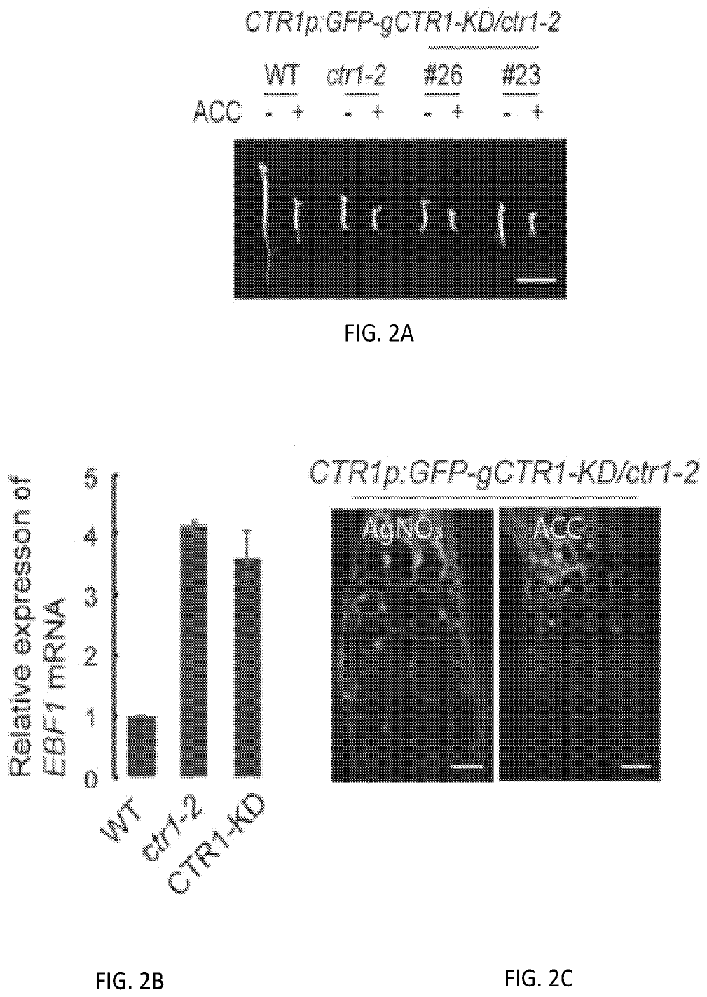

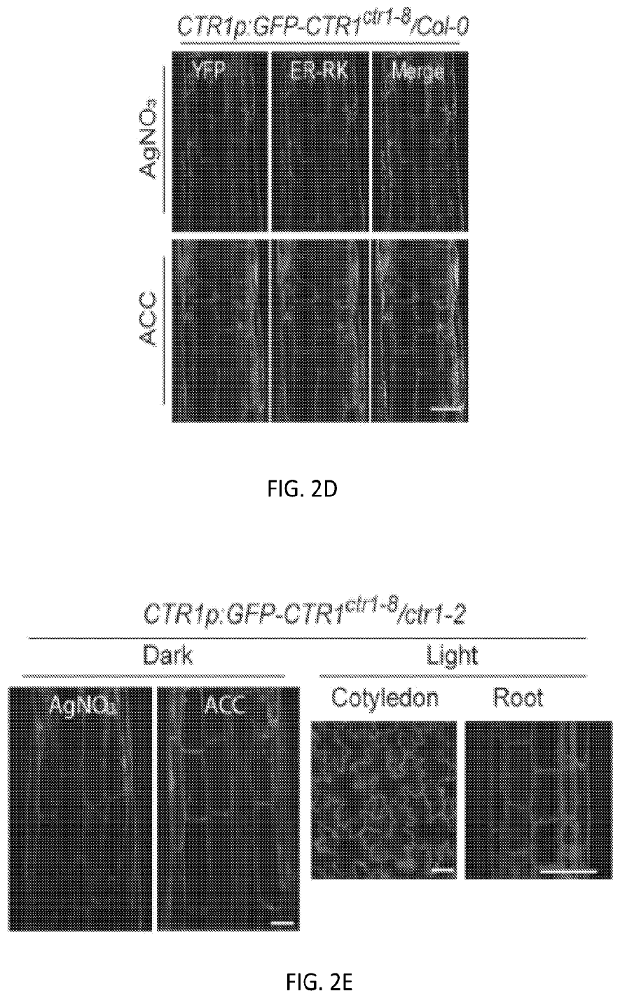

CTR1 interacts with the ETR1 ethylene receptor via its N-terminal domain [10]. Since CTR1 translocation requires the dissociation of CTR1 from the ethylene receptors, we examined whether the N-terminal domain of CTR1 inhibits CTR1 nuclear translocation. To test this, the CTR1 kinase domain (CTR1-KD) lacking the N-terminal domain (FIG. 1A) was expressed in stable transgenic plants from its native promoter. The CTR1p:GFP-gCTR1-KD transgene did not rescue ctr1-2 in etiolated and light grown plants (FIG. 2A). Consistent with this, CTR1p:GFP-gCTR1-KD seedlings had a constitutive expression of EBF1 that was comparable to that in ctr1-2 (FIG. 2B). The failure of CTR1-KD transgene to complement ctr1-2 could result from lack of targeting to the ER and CTR1-KD was indeed constitutively localized to the nucleus, whether expressed from its own or the CaMV 35S promoter (FIG. 2C).

We explored if binding of CTR1 to the ethylene receptors might play a role in its nuclear localization. The ctr1-8 mutation blocks the interaction of CTR1 with ETR1 in a prior yeast-2-hybrid assay [20] but does not affect intrinsic kinase activity. Consistent with the hypermorphic nature of ctr1-8, the GFP-CTR1^{ctr1-8} transgene partially complemented ctr1-2 in both light- and dark-grown seedlings (FIGS. 2B and 2C). Previous fractionation studies demonstrated that ctr1-8 mutant protein was mainly found in a soluble fraction with a minor fraction still in membrane fraction [11], in contrast to the predominant ER localization of wild-type CTR1. Conforming to the prior results, CTR1^{ctr1-8} localized to ER, displaying co-localization with ER marker (FIG. 2D). However, surprisingly, GFP-CTR1^{ctr1-8} did not translocate to the nucleus in either etiolated or light-grown seedlings, regardless of ACC treatment (FIG. 2E). This suggests that interaction with the ethylene receptors is required for nuclear translocation of full-length CTR1, perhaps facilitating a modification of CTR1 in response to ethylene at the ER that relieves the inhibition of nuclear localization by the N-terminal domain.

### The Nuclear Movement of CTR1 is Independent of Kinase Activity

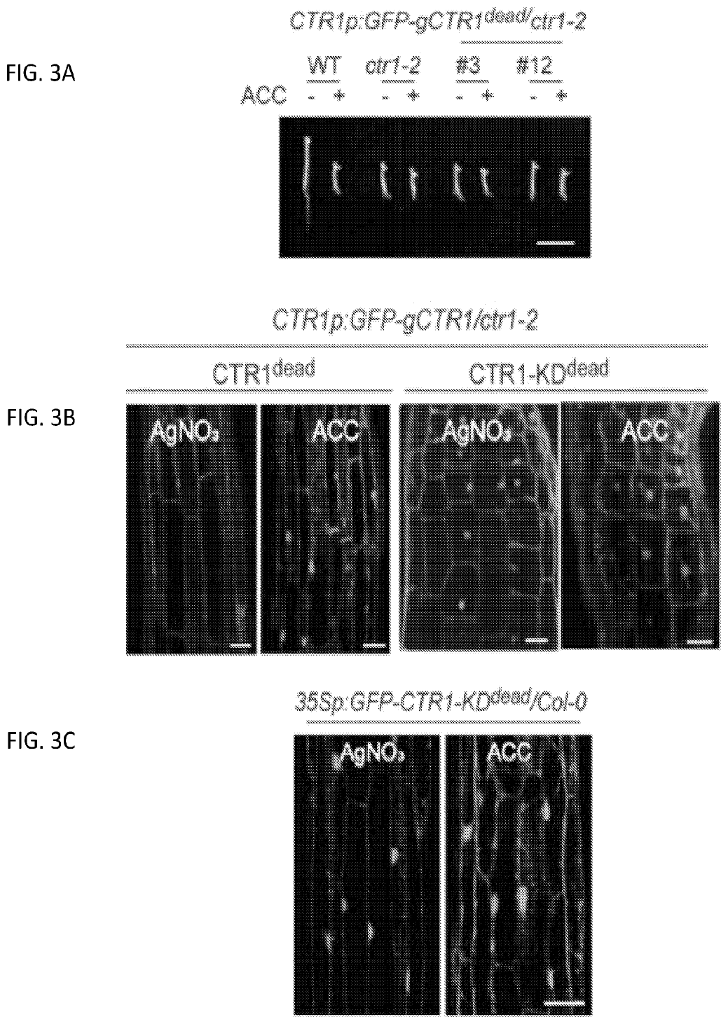

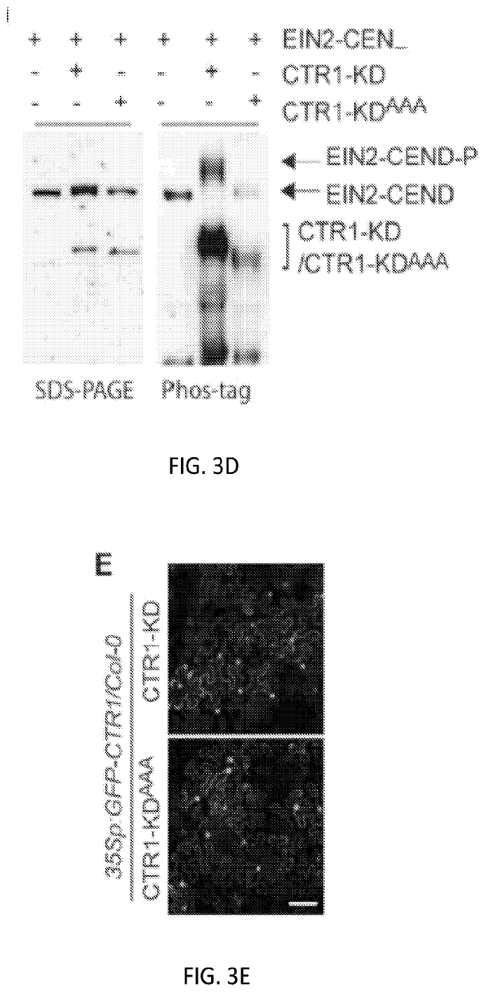

To address the role of kinase activity in CTR1 localization, we expressed a catalytically dead ctr1 mutant (CTR1p:GFP-gCTR1^{dead}) in the ctr1-2 mutant background. The CTR1^{dead} transgene failed to rescue ctr1-2 in either the light or the dark (FIG. 3A), consistent with its hypomorphic nature. Similar to the wild-type, native promoter-driven full-length GFP-CTR1^{dead} translocated to the nucleus upon ACC treatment but GFP-CTR1 under 35S promoter constitutively localized in the nucleus (FIG. 3B). Also consistent with wild-type, CTR1-KD^{dead} expressed under 35S or native promoter (CTR1p:GFP-gCTR1-KD^{dead}/ctr1-2 and 35S:GFP-CTR1^{dead}) were constitutively localized to the nucleus (FIGS. 3B and C). Similar to CTR1-KD, CTR1-KD^{dead} transgene did not rescue ctr1-2 in both dark and light-grown seedlings.

CTR1-KD autophosphorylates on four residues (S703/T704/S707/S710) located within the activation loop, and this autophosphorylation is critical for CTR1 kinase activity and homodimer formation [21]. To test the role of this autophosphorylation, we altered these three residues (T704/S707/S710) to Ala (CTR1^{AAA}), which has been shown to disrupt homodimer formation [21]. We confirmed that CTR1-KD^{AAA} is catalytically inactive using an EIN2 substrate (FIG. 3D). Wild-type CTR1-KD, but not CTR1-KD^{AAA}, phosphorylated EIN2-CEND when co-expressed in Arabidopsis mesophyll protoplasts (FIG. 3D). Consistent with CTR1-KD^{dead}, CTR1-KD^{AAA} was constitutively localized to the nucleus (FIG. 3E). Together, these results indicate that kinase activity is not required for CTR1 nuclear translocation.

### CTR1-Mediated Fast Growth Recovery of Seedlings Following Ethylene-Induced Growth Inhibition

Both ctr1-1 and ctr1-8 mutants show constitutive ethylene responses despite the observation that CTR1^{dead} and CTR1^{ctr1-8} proteins respond differentially to ethylene for nuclear translocation. This indicates that CTR1 nuclear movement does not control the primary ethylene response, but rather may influence ethylene response kinetics by fine-tuning nuclear ethylene signaling. To test this hypothesis, we measured ethylene growth response kinetics of hypocotyls of etiolated seedlings, which has been widely exploited to analyze ethylene mutants [18, 22]. There are two phases of growth inhibition of wild-type Arabidopsis hypocotyls in response to ethylene; phase I begins 10 min after ethylene treatment and was characterized by a rapid deceleration in growth rate. After a transient plateau in growth rate for 15 min, phase II growth inhibition is initiated with a further suppression of growth, lasting 30 min until the growth rate reaches a new, low steady-state rate [18, 22]. Genetic studies have revealed that EIN2 is necessary for both phases, but only phase II requires EIN3/EIL1 [23]. Interestingly, upon removal of ethylene during phase II, hypocotyl growth rapidly recovers to the pre-treatment growth rate within 90 min [18, 22], which indicates the existence of a mechanism to rapidly shut off the ethylene response.

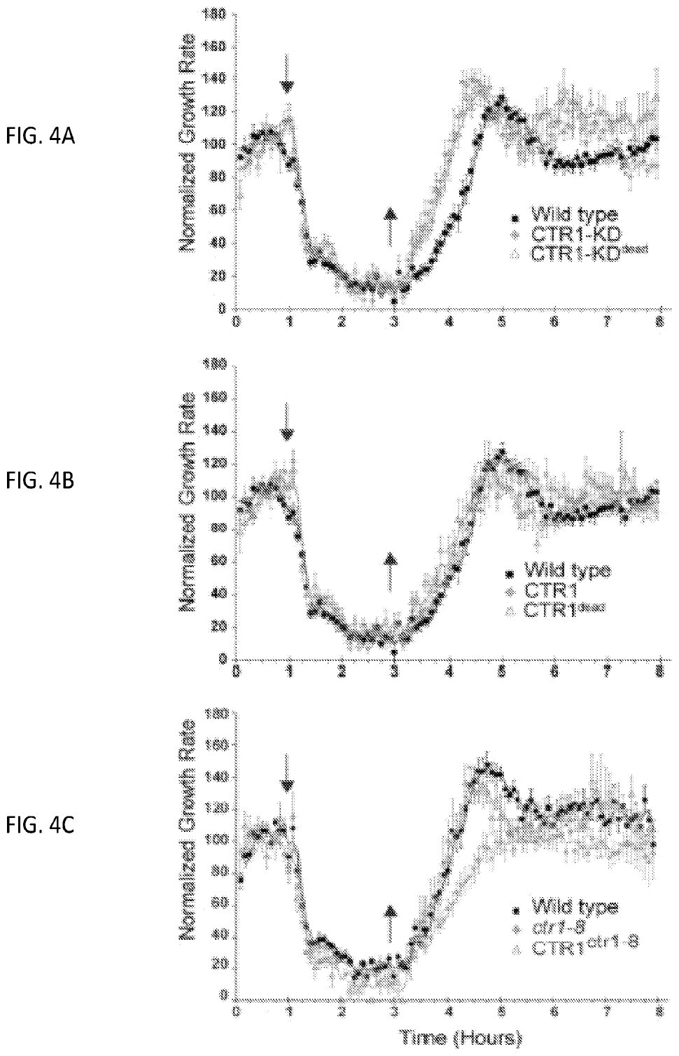

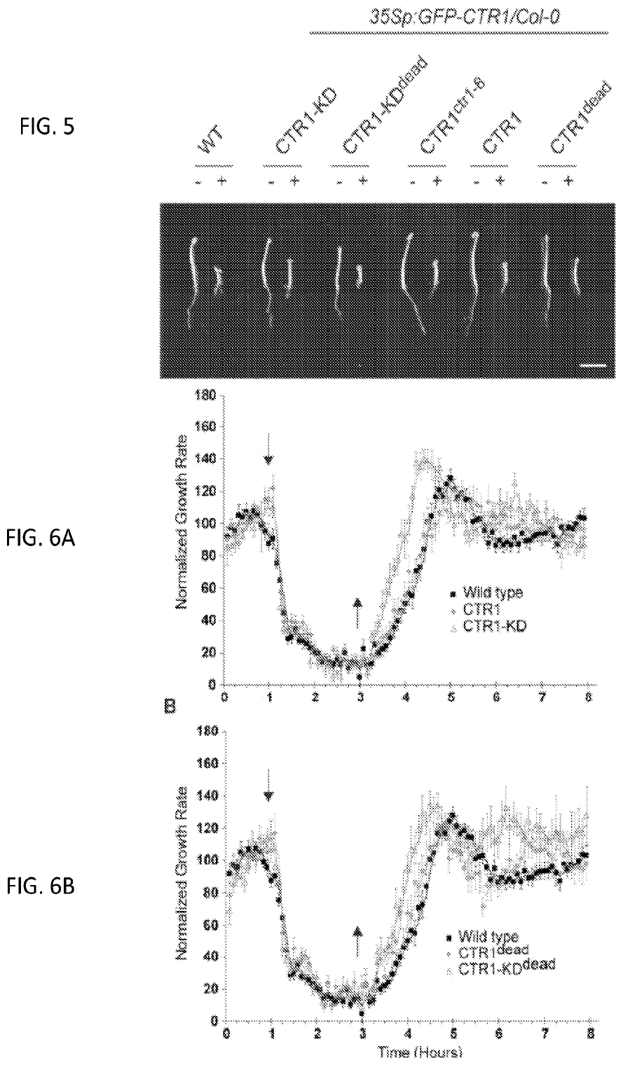

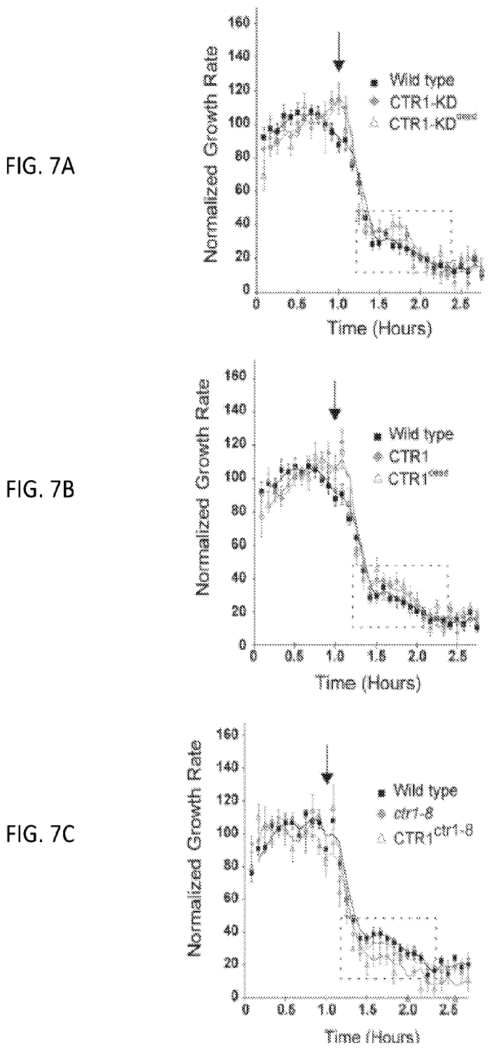

To examine if nuclear-localized CTR1 plays the rapid inhibition or recovery kinetics when ethylene is added or removed, respectively, we performed time-lapse analysis of the ethylene response growth kinetics of seedlings expressing CTR1-KD or CTR1-KD^{dead}, both of which showed strong constitutive nuclear localization. Both KD lines exhibited a comparable triple response to wild-type seedlings in response to ethylene (FIGS. 4A-4C, and FIG. 5). Upon ethylene exposure, both seedlings had a magnitude of growth inhibition similar to wild-type seedlings. However, upon removal of ethylene gas, the recovery of hypocotyl growth rate of both transgenic lines after ethylene removal was substantially faster (˜45 min) than the wild type (FIG. 4A). Seedlings expressing full-length CTR1 or CTR1^{dead} from 35S promoter, both of which showed some levels of constitutive nuclear localization (FIG. 1F and FIG. 5), also exhibited slightly faster growth recovery kinetics than the wild type (FIG. 4B). However, recovery to the basal growth rate was slower than both CTR1-KD and CTR1-KD^{dead} (FIGS. 6A-6B), which may be resulted from its N-terminus-mediated inhibition on full-length CTR1 nuclear movement. Besides, both KD and KD^{dead} seedlings had higher growth rate during the plateau between phase I and II compared to wild type (FIGS. 4A-4B, and 7A-7C), indicating CTR1-mediated suppression on growth inhibition during the phase I plateau.

ctr1-8 displayed comparable growth inhibition response kinetics to that of wild type upon exposure to ethylene (FIG. 4C). However, it recovered substantially slower (˜90 min) than the wild type after removal of ethylene. The delayed hypocotyl growth recovery in ctr1-8 resembled to that observed with EIN3 overexpression or loss of EBF2 reported in previous studies [19]. Interestingly, 35S:GFP-CTR1^{CTR1-8} hypocotyls showed almost identical growth recovery kinetics as the wild type (FIG. 4E), despite that CTR1^{ctr1-8} not being translocated to the nucleus. This is likely due to the existing endogenous CTR1 in CTR1^{ctr1-8}, but not in ctr1-8 mutant seedlings. By contrast to KD and KD^{dead} OE seedlings, both ctr1-8 and 35S:GFP-CTR1^{CTR1-8} hypocotyls displayed the phase I plateau at lower growth rate than wild-type. Together, these results suggest that nuclear-localized CTR1 inhibits EIN2-induced ethylene response, probably via upregulation of EBFs, leading to the transient suppression of ethylene-induced the phase I growth inhibition and fast growth recovery upon removal of ethylene in a mechanism independent of kinase activity.

### CTR1 Promotes Degradation of EIN3 Independent of Kinase Activity

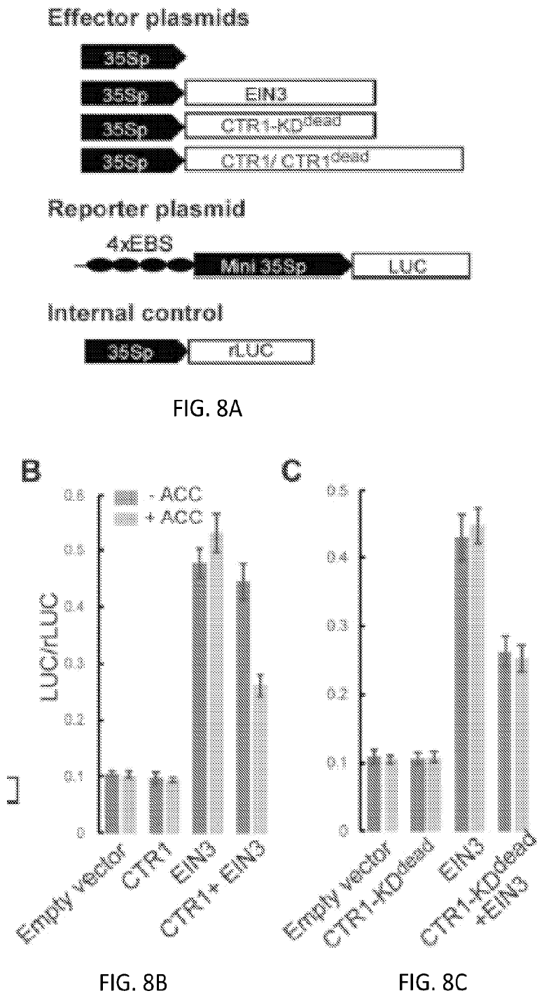

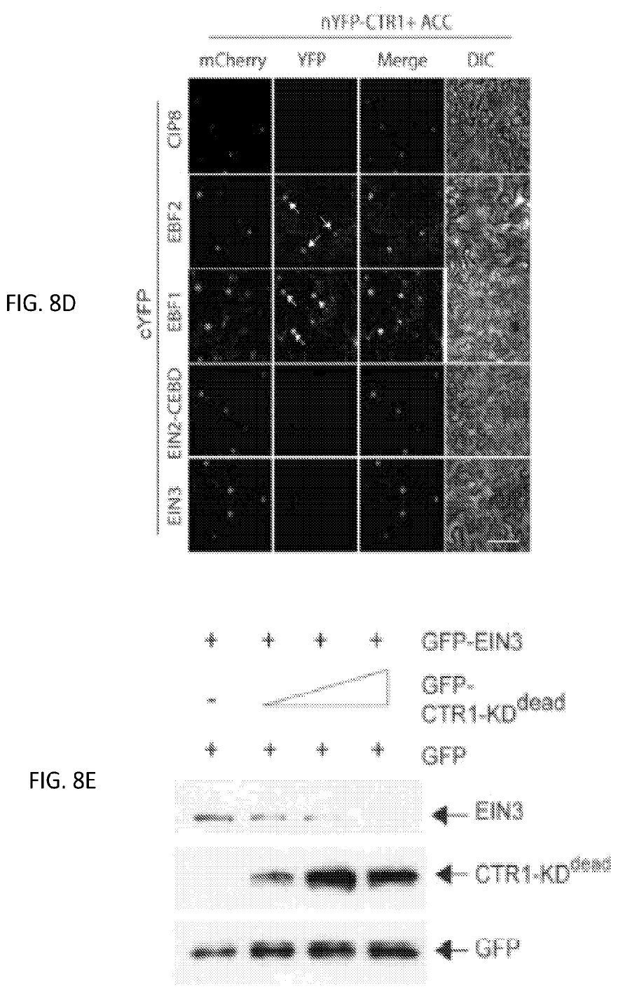

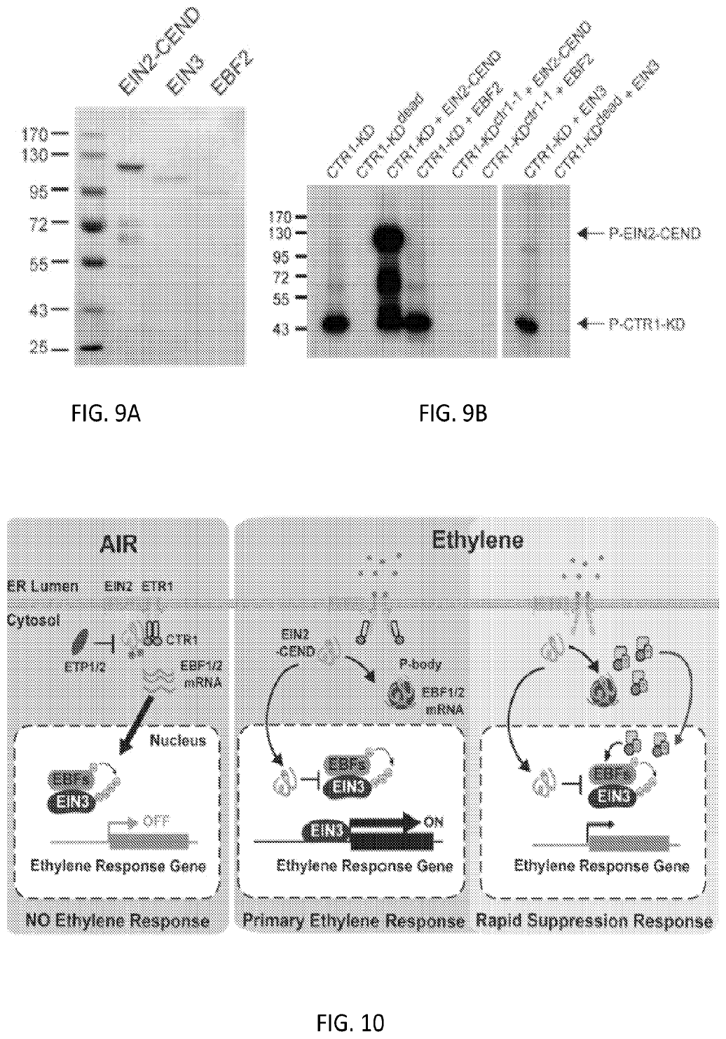

The correlation between nuclear-localized CTR1 and the fast growth recovery kinetics suggests that CTR1 down-regulates nuclear ethylene responses. Thus, we explored if CTR1 modulates EIN3 function in the nucleus in an ethylene-dependent manner. In transiently transformed Arabidopsis protoplasts, the EIN3-dependent activation of a reporter (EBS-LUC) was significantly decreased in an ACC-dependent manner by co-expression with full-length active or inactive CTR1 (CTR1^{dead}) (FIGS. 8A and 8B). Since CTR1 alone did not activate EBS-LUC expression, we ruled out the possibility that CTR1 binds to the EBS. The co-expression of CTR1-KD^{dead} with EIN3 also resulted in reduced EIN3-mediated EBS-LUC activation, but this effect was independent of exogenous ACC (FIG. 8C). To further examine the underlying mechanism, we investigated whether CTR1 directly interacts with EIN3 or other ethylene signaling components in the nucleus using BiFC assays. Wild-type CTR1 did not show interaction with EIN2-CEND as well as a negative control COP-interacting protein 8 (CIP8) in tobacco cells. In the presence of ACC, wild-type CTR1 interacted with EBF1 and 2 (FIGS. 8A and 9A-9B). Wild-type CTR1 also interacted with EBFs in the absence of ACC, probably due to some fraction of the CTR1 translocating to the nucleus owing to the overexpression of CTR1. Furthermore, CTR1-KD^{dead} interacted with EBFs regardless of ACC, but not with EIN2-CEND and EIN3 (FIG. 8D). Together, the results suggest that ethylene receptor-mediated inactivation and conformational changes of CTR1, including the displacement of N-terminus from the kinase domain, is required for CTR1-EBFs interaction and for inhibiting interaction between CTR1 and EIN2-CEND. Subsequent EIN3 stability assay revealed that nuclear-localized CTR1-KD^{dead} stimulates EIN3 degradation, which did not accompany with a reduction on EIN3 transcript (FIGS. 8E and 8F). Half-life experiments showed that EIN3 half-life was substantially shortened with the co-expression of CTR1-KD^{dead} compared to when EIN3 expressed alone in protoplasts (FIG. 8G). Moreover, in vitro kinase assay demonstrated that CTR1 does not phosphorylate EBF2 (FIGS. 9A-9B), reinforcing that CTR1 kinase activity is not involved in the process. Together, these results demonstrated that upon translocation to the nucleus, CTR1 stimulates EBF function via a direct interaction, which results in rapid EIN3 degradation without its kinase activity (FIG. 10).

### The Correlation Between Nuclear-Localized CTR1 and Stress Responses

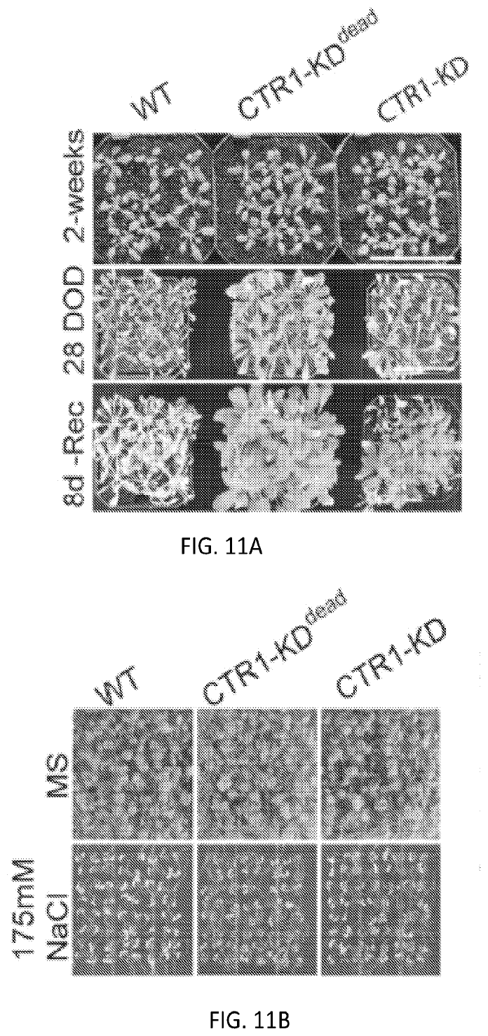

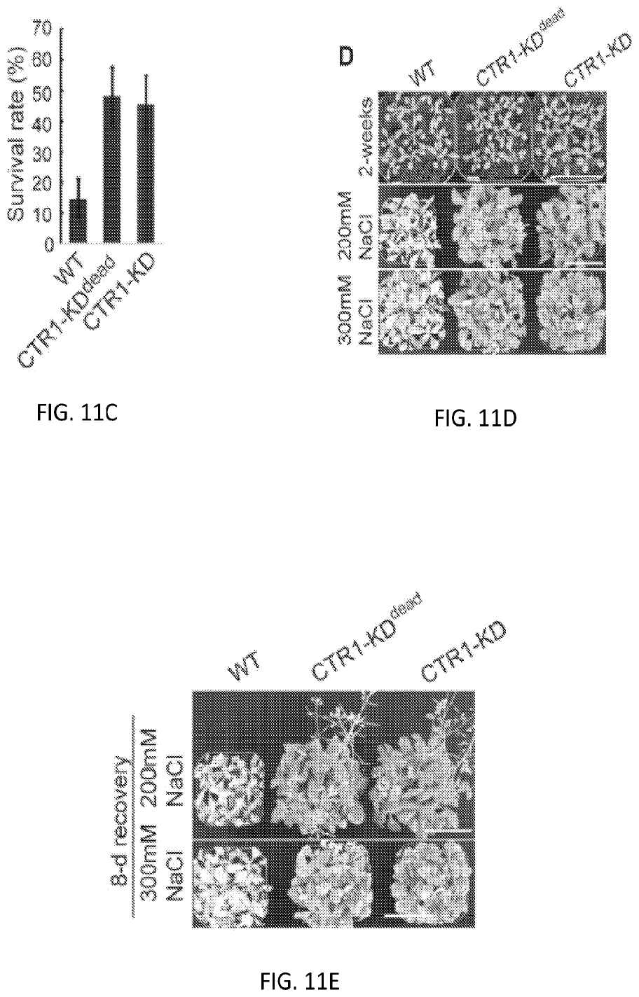

The resiliency to stresses is determined not only by the acclimation to stress, but also timely recovery after the stress removal. Since expression of the CTR1 kinase domain resulted in the dampening of ethylene signaling as well as fast growth recovery after ethylene removal (FIG. 4A), we hypothesized that the CaMV 35S-CTR1-KD^{dead} or -CTR1-KD plants would have enhanced stress tolerance. To examine the effect of water deficit stress, CTR1-KD^{dead}, and CTR1-KD seedlings were subjected to water stress. After 28-d of no watering, wild-type, CTR1-KD^{dead}, and CTR1-KD plants displayed similar levels of the symptom of drought-related stress though CTR1-KD^{dead} plants looked slightly bigger and more turgid compared to wild type and CTR1-KD (FIG. 11A). However, after 8-days of re-watering, the majority of CTR1-KD^{dead} and CTR1-KD plants were recovered from the stress (66% and 50% survival rate, respectively), whereas the wild-type plants were completely dead. Similarly, both CTR1 lines exhibited greater resistance to salt stress. CTR1-KD^{dead} and CTR1-KD seedlings showed higher survival rate (48% and 45%, respectively) than WT (<12%) (FIG. 11C) on medium with NaCl. Further studies on the effects of long-term salt stress on soil reinforced that both lines were highly salt-tolerant. Two-weeks-old wild type, CTR1-KD^{dead}, and CTR1-KD plants were irrigated with 200 or 300 mM NaCl solution for 28-d followed by 8-d after recovery. Unlike the WT seedlings which developed severe growth inhibition with wilting and chlorosis, most of the CTR1-KD^{ctr1-1} and CTR1-KD plants did not show obvious phenotypic changes and growth inhibition (FIGS. 11D and 11E), demonstrating the strong salt-stress tolerance traits of the lines. These results firmly demonstrated that nuclear-localized CTR1 plays a critical role in conferring plants better stress response, likely through diminishing the strength of strong ethylene signaling during the stress acclimation and via CTR1-induced timely growth recovery after stress removal.

FIGS. 11A-11E demonstrate fast growth recovery kinetics confers stress tolerance (A-B) CTR1-KDctr1-1 plants show strong stress tolerance to drought (A) or salinity (B). 2-weeks old WT and CTR1-KDctr1-1 seedlings were subjected for water stress by withdrawing water for 28-d, followed by 7-d re-watering (FIG. 11A). For salt stress, 2-weeks-old seedlings were irrigated with 200 or 300 mM NaCl solution for 24-d. (FIG. 11C) Model for ethylene-induced CTR1 nuclear translocation and suppression of ethylene response. In the absence of ethylene, CTR1 localizes to the ER and phosphorylates EIN2, leading to the proteolytic degradation of EIN2 via ETP1/2. Upon the perception of ethylene by the ethylene receptors, inactivated CTR1 no longer phosphorylates EIN2, resulting in proteolytic cleavage of the C-terminal domain of EIN2, releasing EIN2-CEND. EIN2-CEND is subsequently translocated to the nucleus for activation of EIN3 (1). Likewise, in the presence of ethylene, inactive CTR1 is released from the receptor, which stimulates the nuclear translocation of CTR1 via an unknown mechanism. The nuclear localized CTR1 then binds to EBF1 and 2, activating EBF function thus leading to degradation of EIN3 (2). Proteolytic degradation of EIN2-CEND in the nucleus may also contribute to rapid turn-off of ethylene response during the growth recovery phase. In this process, nuclear movement kinetics of CTR1 is likely slower than EIN2-CEND, and an unknown component whose expression is EIN3/EIL-dependent may involve in the CTR1-mediated downregulation of EIN3 function.

Upon perception of environmental signals via cell surface receptors, the specificity of cellular responses to the stress are mediated by the spatial and temporal dynamics of downstream signaling networks. Here we provide convincing evidence for the identification of a mechanism linking the spatiotemporal regulation of CTR1 to organismal stress responses. We discover that upon inactivation by ethylene, CTR1 re-locates from the ER to the nucleus where CTR1 downregulates the nuclear ethylene responses. Remarkably, we further discovered that the nuclear movement of CTR1 is tightly associated with a plant's resilience to stress, which presents a potential key to manipulating plant's adaptability to abiotic stresses such as drought and salinity.

Despite the constitutive nuclear localization of CTR1, the hypocotyls of CTR1 OE did not initiate growth recovery until ethylene gas was purged (FIGS. 4A and B). One likely scenario for this phenomenon is that the tight regulation on equilibrium between EIN2-mediated EIN3 activation and CTR1-mediated EIN3 inhibition. CTR1 only visibly amasses in the nucleus approximately 30 min after ethylene treatment (FIG. 1H), which is coincident with the initiation time of the first plateau after the phase I inhibition and is slower than accumulation of EIN2. Similar to wild type hypocotyls, the growth rate of hypocotyls of CTR1 OE seedlings reached to the phase I plateau about 30 min after ethylene exposure (FIGS. 4A-4C), but interestingly they have a higher growth rate than the wild type during the plateau (FIGS. 4A-4C and 7A-7C). These results indicate that overexpression of nuclear-localized CTR1 hampers EIN2-induced activation of ethylene responses beyond the levels normally inhibited by endogenous CTR1 during this plateau. Intriguingly, ebf1-3 loss-of-function mutant did not have this plateau in growth rate, but rather immediately entered phase II growth inhibition [19]. However, due to the continued binding of ethylene to the receptors, the strength of EIN2-induced activation of EIN3 would overcome the effects of CTR1-medated downregulation of EIN3 in the presence of ethylene, thus promoting continued growth suppression. Upon removing ethylene gas, the equilibrium may rapidly shift from EIN2-mediated activation of EIN3 to CTR1-mediated EIN3 degradation, initiating rapid growth recovery. Investigation of EIN2-CEND stability or activity during the short window after purging ethylene gas will provide an answer to this question. Moreover, the ctr1-8 mutant seedlings still showed some levels of growth recovery despite its complete failure to translocate to the nucleus in response to ethylene (FIG. 4C). These results imply the existence of other recovery mechanisms that are not dependent on CTR1 for controlling the fast growth recovery such as ethylene-receptor-mediated inactivation of ethylene responses [23].

Due to the lack of canonical nuclear localization sequences, how CTR1 translocates from the ER to the nucleus and whether other ethylene-activated components are involved in the trans-localization process remains to be determined. Moreover, elucidation of the CTR1-independent mechanism that regulates the fast growth recovery kinetics will bring more insights into the mechanistic understanding of ethylene response regulation. Ethylene interacts with a myriad of internal and external stimuli, regulating plant growth and stress responses. Thus, it will be of great interest to revisit crosstalk with other pathways taking into consideration the nuclear role of CTR1.

### Materials and Methods

### Plant Materials and Growth Conditions

Arabidopsis Thaliana Col-0 was used as the wild-type reference throughout the study. All plants were grown in either long-day or short-day conditions at 22° C.±2° C. or in vitro on Murashige and Skoog (MS) Basal Medium supplemented with 0.8% plant agar (pH 5.7) in continuous light chamber at 21° C. All plants used were homozygous or T2 crosses. Homozygous transgenic lines were identified by segregation of antibiotics resistance followed by confirmation of protein expression via western blot analysis.

### CTR1 Constructs and Site-Directed in Vitro Mutagenesis for Arabidopsis Transformation

All molecular cloning was performed using the Gateway (Invitrogen) or infusion cloning strategies otherwise specified. To create CTR1p-YFP-gCTR1, we PCR-amplified three separate overlapping fragments (CTR1 promoter, YFP, and genomic fragment of CTR1). 0.96 kb of CTR1 promoter and 4.7 kb of full length CTR1 genomic fragment were amplified using Col-0 genomic DNA as a template and the YFP coding sequences was amplified using binary vector pEarleyGate 104. Three overlapping fragments were subsequently subjected to infusion reaction with Stu1 and Xba1 digested pEarleyGate 104, creating the full length CTR1 clone in pEarleyGate 104 backbone. Mutations on genomic CTR1 fragment (G354E ctr1-8 or D694E ctr1-1 mutation) were introduced by overlapping PCR using primers possessing mutations and the resulting fragments were used for infusion reactions as described above. CTR1p-GFP-gCTR1 and its mutant variant constructs were also constructed in an identical manner as above except using GFP instead of YFP. To construct 35p:GFP-CTR1, the coding sequences of full length or kinase domain of CTR1 was cloned into pENTR entry vector and was subsequently transferred to binary vector pSITE2CA. Mutations were introduced in the coding sequences of CTR1 in pENTR vector and the sequences were further transferred to pSITE2CA.

### Bimolecular Fluorescence Complementation Constructs

Following coding sequences with stop were transferred from pENTR vector into pCL112 to generate N-terminal nYFP-fusion: CTR1, CTR1^{dead}, CTR1^{ctr1-8}, CTR1-KD^{dead}.

Following coding sequences without stop were transferred from pENTR vector into pBAT-YFPc to generate C-terminal cYFP-fusion: ETR1, EIN3, EIN2-CEND, and CIP8. The coding sequences of EBF1 and EBF2 in pENTR was transferred into pCL113, creating cYFP-EBF2.

### Transactivation Assay

To construct effector plasmids, the coding sequences of EIN3, CTR1^{dead}, or CTR1-KD^{dead} in pENTR gateway vector were transferred into pEarleyGate 203, creating a myc-tagged fusion protein. To generate a reporter plasmid, we PCR-amplified 4×EBS with minimal 35S promoter using EBS-GUS-pCAMBIA-1381Z as a template. The coding sequences of Luciferase was PCR-amplified using pEGB 35S:Luciferase:Tnos (Addgene, GB0110). The amplified two fragments and StuI and XbaI-digested pEarleyGate 104 vector were further subject to infusion reaction to create a 4×EBS: min35Sp-Luciferase. All final plasmid constructs were verified by DNA sequencing. We used pEGB 35S:Renilla:Tnos (Addgene, GB0109) as an internal control plasmid. To perform transactivation assay, Arabidopsis mesophyll protoplasts were isolated from 3 weeks old Arabidopsis grown under a 12 h light/12-h dark regime at 22° C. as previously described. For transfection, 5×10⁵ of protoplasts were incubated with the appropriate effector (5 μg) and reporter plasmid DNA (2.5 μg) in 20% polyethylene glycol (Sigma-Aldrich) for 5 min. Transfected protoplasts were then washed twice with W5 solution and incubated for 12-16 h followed by lysis with Cell Culture Lysis Solution (Promega). The activity of LUC and rLUC with cell lysate were measured using a Dual Luciferase Assay System (Promega).

### Phostag Gel Analysis

Preparation of Phos-Tag polyacrylamide gels and subsequent immunoblot was performed as described in the manufacture's instruction. Phos-Tag gel was prepared using 10 ml of 8% acrylamide and X μM Phos-tag for the resolving gel and 4 mL of 4.5% acrylamide for the stacking gel. The gel was run initially at X V for 30 min at RT then transferred to 30 V for 16 h at 4° C. using 1× running buffer (50 mM Tris base pH8.3, 0.1% SDS, 192 mM glycine). The resolving gel was then soaked in 2×100 mL of transfer buffer (192 mM glycine, 25 mM Tris-base pH 8.0, and 10% methanol containing 10 mM EDTA) for 30 min each time and then washed once for 20 min in transfer buffer without EDTA. The gel was then further incubated in 100 mL transfer buffer containing 0.2% (w/v) SDS (two 20-min incubations). Protein was blotted onto Nitrocellulose membrane by semi-dry procedure at X V (at constant voltage) for X h at RT. Membranes were blocked using 5% nonfat milk and probed with 1:2000 dilution of Roche Anti-GFP (SigmaAldrich Cat #. 11814460001) and 1:20000 dilution of anti-mouse HRP secondary antibody. Regular SDS-PAGE analysis was conducted as previously described.

### Time-Lapse Growth Recovery Analysis

To measure ethylene response growth kinetics of seedlings, seedlings were grown on vertically orientated petri plates in darkness to a height of 3 to 4 mm (42-46 h) before the beginning of growth-rate measurements. The agar plates were placed vertically in a holder and fitted with a lid for continuous gas flow (100 mL min⁻¹). Seedlings were grown in air for 1 h followed by application of ethylene (typically 1 or 10 ppm) for 2 h, followed by removal of ethylene. Images were acquired every 5 min using a CCD camera fitted with a close-focus lens with illumination provided by infrared LEDs. The growth rate of the hypocotyls in every time interval was then calculated. Under these conditions, the equilibration time of the chamber at these flow rates was approximately 30 sec, which was much faster than the image acquisition time. From this, we determined how various mutants affect growth inhibition kinetics when ethylene was added and recovery kinetics when it was removed to give information about how these signaling components affect whole organism responses.

### Yeast-2-Hybrid

The coding sequences of the full-length CTR1 or kinase domain of CTR1 (CTR1-KD) with or without ctr1-1 mutation (D694E) in pENTR GW entry vector were transferred into pGBDT7 or pGADT7. The resulting bait clone were paired with EIN2-CEND, EIN3, EBF1, or EBF2 clone in pGBDT7 or pGADT7 vector and tested their interaction in yeast. Positive interactions between prey and bait were selected on medium lacked histidine, tryptophan, leucin contained X mM 3-aminotrizole (3-AT). Due to autoactivation of full-length CTR1 in pGBDT7, CTR1-KD or CTR1KD^{dead} was used to generate CTR1 bait construct.

### Confocal Microscopy

All imaging of GFP, YFP, mCherry, and RFP were carried out using a laser-scanning confocal microscope (Zeiss LSM880 upright). Samples were directly mounted on a glass slides in water. For imaging of Arabidopsis seedlings transformed with CTR1 constructs, seedlings were grown on MS media supplemented with or without 10 μM AgNO3 in dark for 3-days. For ACC treatment, seedlings on MS without AgNo3 were treated with 200 μM ACC dissolved in water for 2 hr. For ethylene treatment, seedlings were directly gown on MS media in GC-vials for 3 days. The GC vials subsequently were capped and injected with 1 ppm ethylene gas and incubated for 2 h. For nuclear imaging, 3-day-old dark grown seedlings were treated with 200 μM ACC in combination with x μM Hoechst33342 for X h, followed by brief washing before examination. For light grown seedlings imaging, Arabidopsis seedlings were grown on MS in light for 5 days in light and used for imaging analysis. For imaging of fluorescence signals in protoplasts, transfected protoplasts were incubated with 200 μM ACC for 2 h in dark and subjected to examination. All imaging was performed using more than 3 independent biological replicates of at least X independent lines. To image BiFC, leaf disks of infiltrated tobacco leaves with CTR1 and counterpart constructs (ETR1, EIN2-CEND, EIN3, EBF, and CIP8) was mounted on a glass slide in water and examined the interaction.

### Arabidopsis Drought and Salt Stress Experiments

For the drought recovery experiments, Arabidopsis seedlings were grown on soil in short-day conditions for 14-days and withdrawn water for 28 days followed by re-watering for 8 days.

To test germination efficiency of seedlings, seedlings were grown on MS media with X % PEG8000 for two weeks and scored the germinated seedlings. For the salt stress treatment, Arabidopsis seedlings were grown on soil in short-day conditions for 14 days. Starting on day 14th, the plants were treated with 200 or 300 mM NaCl for 28 days followed by 8-d of recovery. To test salt tolerance of Arabidopsis seedlings on plates, seedlings were grown on MS medium with 175 mM NaCl for 3 weeks in short-day conditions and the survival rate of the seedlings were scored. The experiment was repeated three times with similar results.

### In Vitro Kinase Assay

A total of 20 ng purified His₆-CTR1-KD^{WT} or His₆-CTR1-KD^{ctr1-1} protein was incubated with 100 ng of His₆-EIN2^{WT}-His₆, His₆-EBF2, or His₆-EIN3 in kinase reaction buffer [50 mM Tris (pH 7.5), 10 mM MgCl₂, 1× Roche Complete Protease Inhibitor mixture, 1 μCi [γ-³²P]ATP] for 30 min at room temperature. After incubation, reactions were terminated by boiling in 6× Laemmli SDS sample buffer for 3 min. Samples were subjected to SDS/PAGE, dried, and visualized by autoradiography.

### Real-Time Quantitative PCR Analysis of EBF1 mRNA Abundance

Total RNA was prepared from 3d-old Arabidopsis seedlings using RNeasy Plant Mini Kit (QIAGEN) and reverse transcribed using SuperScript II reverse transcriptase (Invitrogen) according to the manufacturers' instructions. Quantitative RT-PCR was performed using PowerUP™ SYBRGreen Master Mix (Applied Biosystems). Primers used are listed in Table S1. Three biological replicates were analyzed with three technical replicates per sample. The relative expression for candidate genes was normalized to ß-tubulin.

### EIN3 Half-Life Experiment

Protoplasts were transfected with myc-tagged EIN3 with or without myc-tagged CTR1-KD^{dead} using polyethylene glycol-mediated method. GFP plasmid was co-transfected as a transfection control and the difference in total amount of plasmid was complemented by adding empty vector. Transfected protoplasts were incubated for 16 h, followed by further incubation with 250 μM cycloheximide (Sigma-Aldrich). Protoplasts were then harvested at different time points for immunoblotting analysis.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that that the scope of the present methods and compositions be defined by the following claims. However, it must be understood that this disclosure may be practiced otherwise than is specifically explained and illustrated without departing from its spirit or scope.

### REFERENCES CITED

1. Abeles, F.B., Morgan, P.W., and Saltveit, M.E.J. (1992). Ethylene in plant biology. San Diego, CA: Academic Press.
2. Bleecker, A.B., and Kende, H. (2000). Ethylene: a gaseous signal molecule in plants. Annu. Rev. Cell. Dev. Biol. 16, 1-18.
3. Alonso, J.M., and Ecker, J.R. (2001). The ethylene pathway: a paradigm for plant hormone signaling and interaction. Science's STKE: signal transduction knowledge environment 2001, re1.
4. Hattori, Y., Nagai, K., Furukawa, S., Song, X.J., Kawano, R., Sakakibara, H., Wu, J., Matsumoto, T., Yoshimura, A., Kitano, H., et al. (2009). The ethylene response factors SNORKEL1 and SNORKEL2 allow rice to adapt to deep water. Nature 460, 1026-1030.
5. Lee, H.Y., Chen, Z., Zhang, C., and Yoon, G.M. (2019). Editing of the OsACS locus alters phosphate deficiency-induced adaptive responses in rice seedlings. J. Exp. Bot. 70, 1927-1940.
6. Song, L., and Liu, D. (2015). Ethylene and plant responses to phosphate deficiency. Frontiers in plant science 6, 796.
7. Thao, N.P., Khan, M.I., Thu, N.B., Hoang, X.L., Asgher, M., Khan, N.A., and Tran, L.S. (2015). Role of Ethylene and Its Cross Talk with Other Signaling Molecules in Plant Responses to Heavy Metal Stress. Plant Physiol. 169, 73-84.
8. Alonso, J.M., Hirayama, T., Roman, G., Nourizadeh, S., and Ecker, J.R. (1999). EIN2, a bifunctional transducer of ethylene and stress responses in Arabidopsis. Science (New York, N.Y.) 284, 2148-2152.
9. Bisson, M.M., Bleckmann, A., Allekotte, S., and Groth, G. (2009). EIN2, the central regulator of ethylene signalling, is localized at the ER membrane where it interacts with the ethylene receptor ETR1. Biochem. J. 424, 1-6.
10. Clark, K.L., Larsen, P.B., Wang, X., and Chang, C. (1998). Association of the Arabidopsis CTR1 Raf-like kinase with the ETR1 and ERS ethylene receptors. Proceedings of the National Academy of Sciences of the United States of America 95, 5401-5406.
11. Gao, Z., Chen, Y.F., Randlett, M.D., Zhao, X.C., Findell, J.L., Kieber, J.J., and Schaller, G.E. (2003). Localization of the Raf-like kinase CTR1 to the endoplasmic reticulum of Arabidopsis through participation in ethylene receptor signaling complexes. J. Biol. Chem. 278, 34725-34732.
12. Ju, C., Yoon, G.M., Shemansky, J.M., Lin, D.Y., Ying, Z.I., Chang, J., Garrett, W.M., Kessenbrock, M., Groth, G., Tucker, M.L., et al. (2012). CTR1 phosphorylates the central regulator EIN2 to control ethylene hormone signaling from the ER membrane to the nucleus in Arabidopsis. Proceedings of the National Academy of Sciences of the United States of America 109, 19486-19491.
13. Kieber, J.J., Rothenberg, M., Roman, G., Feldmann, K.A., and Ecker, J.R. (1993). CTR1, a negative regulator of the ethylene response pathway in Arabidopsis, encodes a member of the raf family of protein kinases. Cell 72, 427-441.
14. Qiao, H., Shen, Z., Huang, S.S., Schmitz, R.J., Urich, M.A., Briggs, S.P., and Ecker, J.R. (2012). Processing and subcellular trafficking of ER-tethered EIN2 control response to ethylene gas. Science (New York, N.Y.) 338, 390-393.
15. Wen, X., Zhang, C., Ji, Y., Zhao, Q., He, W., An, F., Jiang, L., and Guo, H. (2012). Activation of ethylene signaling is mediated by nuclear translocation of the cleaved EIN2 carboxyl terminus. Cell Res. 22, 1613-1616.
16. Merchante, C., Brumos, J., Yun, J., Hu, Q., Spencer, K.R., Enriquez, P., Binder, B.M., Heber, S., Stepanova, A.N., and Alonso, J.M. (2015). Gene-specific translation regulation mediated by the hormone-signaling molecule EIN2. Cell 163, 684-697.
17. Li, W., Ma, M., Feng, Y., Li, H., Wang, Y., Ma, Y., Li, M., An, F., and Guo, H. (2015). EIN2-directed translational regulation of ethylene signaling in Arabidopsis. Cell 163, 670-683.
18. Binder, B.M., O'Malley R, C., Wang, W., Moore, J.M., Parks, B.M., Spalding, E.P., and Bleecker, A.B. (2004). Arabidopsis seedling growth response and recovery to ethylene. A kinetic analysis. Plant Physiol. 136, 2913-2920.
19. Binder, B.M., Walker, J.M., Gagne, J.M., Emborg, T.J., Hemmann, G., Bleecker, A.B., and Vierstra, R.D. (2007). The Arabidopsis EIN3 binding F-Box proteins EBF1 and EBF2 have distinct but overlapping roles in ethylene signaling. Plant Cell 19, 509-523.
20. Huang, Y., Li, H., Hutchison, C.E., Laskey, J., and Kieber, J.J. (2003). Biochemical and functional analysis of CTR1, a protein kinase that negatively regulates ethylene signaling in Arabidopsis. Plant J. 33, 221-233.
21. Mayerhofer, H., Panneerselvam, S., and Mueller-Dieckmann, J. (2012). Protein kinase domain of CTR1 from Arabidopsis thaliana promotes ethylene receptor cross talk. J. Mol. Biol. 415, 768-779.
22. Binder, B.M. (2017). Time-Lapse Imaging to Examine the Growth Kinetics of Arabidopsis Seedlings in Response to Ethylene. Methods in molecular biology (Clifton, N.J.) 1573, 211-222.
23. Binder, B.M., Mortimore, L.A., Stepanova, A.N., Ecker, J.R., and Bleecker, A.B. (2004). Short-term growth responses to ethylene in Arabidopsis seedlings are EIN3/EIL1 independent. Plant Physiol. 136, 2921-2927.

1. A method for improving a plant's stress tolerance and a speedy recovery to growth from said stress by promoting constitutive expression of an analog, a fragment, or a mutant of the constitutive triple response 1 (CTR1) protein kinase (SEQ ID NO: 1) in the cell nucleus of said plant.

2. The method according to claim 1, wherein said analog, fragment, or mutant of CTR1 protein kinase (SEQ ID NO: 1) in the cell nucleus of said plant has no protein kinase activity.

3. The method according to claim 1, wherein said analog, fragment, or mutant of CTR1 protein kinase (SEQ ID NO: 1) in the cell nucleus of said plant is a fragment of CTR1 with the N-terminal domain removed (delta N).

4. (canceled)

5. (canceled)

6. (canceled)

7. The method according to claim 1, wherein said CTR1 protein kinase is a mutant (D694E) having SEQ ID NO: 2, a functional analog, or a fragment thereof.

8. The method according to claim 1, wherein said CTR1 protein kinase comprises SEQ ID NO: 3, a functional analog, or a fragment thereof.

9. The method according to claim 1, wherein said CTR1 protein kinase mutant comprises SEQ ID NO: 4, a functional analog, or a fragment thereof.

10. The method according to claim 1, wherein said CTR1 protein kinase mutant with T704A, S707A, and S710A mutations, comprises SEQ ID NO: 5, a functional analog, or a fragment thereof.

11. (canceled)

12. The method according to claim 1, wherein said stress comprises drought, salinity, and other environmental stresses comprising flooding (submergence), freezing, chilling, extreme temperature (cold, frost, heat), radiation, biotic stresses caused by a living organism selected from the group consisting of viruses, bacteria, fungi, nematodes, insects, and arachnids.

13. The method according to claim 1, wherein said plant expresses CTR1-like protein kinase and wherein overexpression of the corresponding CTR1-like genes in the nucleus increases said plant's tolerance to physical and environmental stresses.

14. The method according to claim 1, wherein said plant comprises soybean, corn, rice, sorghum, potato, wheat, barley, and peanut.

15. An analog, a fragment, or a mutant of CTR1 protein kinase (SEQ ID NO: 1) useful for improving a plant's stress tolerance and a speedy recovery to growth from a stress.

16. The analog, a fragment, or a mutant of CTR1 protein kinase (SEQ ID NO: 1) according to claim 15, wherein said CTR1 protein kinase mutant comprises a SEQ ID NO: 2, or a functional analog and/or fragment thereof.

17. The analog, a fragment, or a mutant of CTR1 protein kinase (SEQ ID NO: 1) according to claim 15, wherein said CTR1 protein kinase mutant comprises a SEQ ID NO: 3, or a functional analog and/or fragment thereof.

18. The analog, a fragment, or a mutant of CTR1 protein kinase (SEQ ID NO: 1) according to claim 15, wherein said CTR1 protein kinase mutant comprises a SEQ ID NO: 4, or a functional analog and/or fragment thereof.

19. The analog, a fragment, or a mutant of CTR1 protein kinase (SEQ ID NO: 1) according to claim 15, wherein said CTR1 protein kinase mutant comprises a SEQ ID NO: 5, or a functional analog and/or fragment thereof.

20. The analog, a fragment, or a mutant of CTR1 protein kinase (SEQ ID NO: 1) according to claim 15, wherein said stress comprises both physical and environmental stresses.

21. The analog, a fragment, or a mutant of CTR1 protein kinase (SEQ ID NO: 1) according to claim 15, wherein said stress comprises drought, salinity, and other environmental stresses comprising flooding (submergence), freezing, chilling, extreme temperature (cold, frost, heat), radiation, biotic stresses caused by a living organism selected from the group consisting of viruses, bacteria, fungi, nematodes, insects, and arachnids.

22. The analog, a fragment, or a mutant of CTR1 protein kinase (SEQ ID NO: 1) according to claim 15, wherein said analog, fragment, or mutant of the CTR1 protein kinase (SEQ ID NO: 1) has its N-terminal domain removed.

23. The analog, a fragment, or a mutant of CTR1 protein kinase (SEQ ID NO: 1) according to claim 15, wherein said plant comprises soybean, corn, rice, sorghum, potato, wheat, barley, peanut, and others.

24. An engineered plant or a plant cell, wherein said plant or plant cell expresses or overexpresses an analog, a fragment, or a mutant of CTR1 protein kinase (SEQ ID NO: 1) useful for improving a plant's stress tolerance and a speedy recovery to growth from a stress.
